(19) United States
(12) Patent Application Publication
(10) Pub. No.: US 2014/0113942 A1
(43) Pub. Date: Apr. 24, 2014

(54) PROCESS FOR THE PREPARATION AND PURIFICATION OF AZILSARTAN MEDOXOMIL

(71) Applicant: CADILA HEALTHCARE LIMITED, Ahmedabad (IN)

(72) Inventors: Shriprakash Dhar DWIVEDI, Ahmedabad (IN); Kumar Kamlesh SINGH, Ahmedabad (IN); Jitendra Maganbhai GAJERA, Ahmedabad (IN)

(73) Assignee: CADILA HEALTHCARE LIMITED, Ahmedabad (IN)

(21) Appl. No.: 14/051,572

(22) Filed: Oct. 11, 2013

(30) Foreign Application Priority Data
Oct. 12, 2012 (IN) 3002/MUM/2012

(51) Int. Cl. C07D 413/10; C07D 413/14

(52) U.S. Cl. CPC C07D 413/10; C07D 413/14
USPC 514/364; 548/132

(57) ABSTRACT

The present invention relates to process an improved process for the preparation of azilsartan medoxomil. In particular, the field of invention relates to a process for purification of azilsartan medoxomil. More particularly, the invention relates to an improved process for preparation of azilsartan medoxomil and its pharmaceutically acceptable salts.

### FIELD OF THE INVENTION

The field of the invention relates to process an improved process for the preparation of azilsartan medoxomil. In particular, the field of invention relates to a process for purification of azilsartan medoxomil. More particularly, the invention relates to an improved process for preparation of azilsartan medoxomil and its pharmaceutically acceptable salts.

### BACKGROUND OF THE INVENTION

The following discussion of the prior art is intended to present the invention in an appropriate technical context and allow its significance to be properly appreciated. Unless clearly indicated to the contrary, however, reference to any prior art in this specification should be construed as an admission that such art is widely known or forms part of common general knowledge in the field.

Azilsartan medoxomil potassium (CAS 863031-24-7) is angiotensin II receptor blocker (ARB) that lowers blood pressure by blocking the action of angiotensin II, a vasopressor hormone that constricts blood vessels. When the angiotensin II receptor is blocked, blood vessels stay relaxed and open and blood pressure can be reduced. It is available under the trade name of Edarbi® for the treatment of hypertension or high blood pressure in the recommended dose of 40 mg taken once daily and escalation to 80 mg per day as necessary.

U.S. Pat. No. 7,157,584 B2 (the US '584 patent) discloses the preparation of benzimidazole derivatives, including azilsartan medoxomil potassium, and the use thereof as angiotensin II antagonist. The chemical name of azilsartan medoxomil potassium is (5-Methyl-2-oxo-1,3-dioxol-4-yl)methyl 2-ethoxy-1-{[2′-(5-oxo-4,5-dihydro-1,2,4-oxadiazol-3-yl)biphenyl-4-yl]methyl}-1H-benzimidazole-7-carboxylate monopotassium salt or 1H-Benzimidazole-7-carboxylic acid, 1-[[2′-(2,5-dihydro-5-oxo-1,2,4-oxadiazol-3-yl)[1,1′-biphenyl]-4-yl]methyl]-2-ethoxy-(5-methyl-2-oxo-1,3-dioxol-4-yl)methylester, potassium salt, compound of Formula (I′).

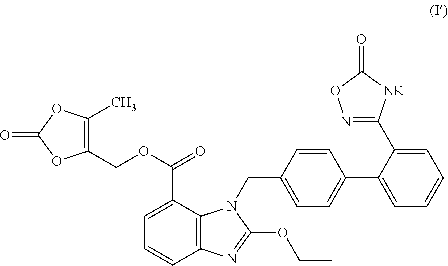

The US '584 patent discloses the process for the preparation of azilsartan medoxomil potassium by reacting a reactive derivative of 2-ethoxy-1-{[2′-(5-oxo-4,5-dihydro-1,2,4-oxadiazol biphenyl-4-yl]methyl}-1H-benzimidazole-7-carboxylic acid or a salt thereof with a compound represented by the Formula

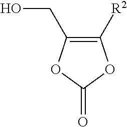

wherein R² can be independently hydrogen atom or C₁₋₆ alkyl, or a salt thereof.

U.S. Pat. No. 5,583,141 and J. Med. Chem. Vol. 39(26) pg. 5228-5235 discloses the process for the preparation of azilsartan. The compound azilsartan medoxomil potassium salt of Formula (I′) have structural similarity with losartan potassium, candesartan cilexetil or olmesartan medoxomil. The structure having acidic group such as tetrazolyl group, a carboxyl group and the biphenyl side chain are common characteristics of angiotensin II antagonists.

U.S. Pat. No. 5,138,069 discloses losartan potassium and process for its preparation. U.S. Pat. No. 5,196,444 discloses candesartan cilexetil and process for its preparation. U.S. Pat. No. 5,616,599 discloses olmesartan medoxomil and process for its preparation.

J. Med. Chem. Vol. 39, p. 625 (1996) discloses the clinical studies of pharmaceutical compound having structural characteristics like losartan, candesartan cilexetil, olmesartan medoxomil and the like.

International (PCT) publication WO 2011/063764 A1 discloses azilsartan organic amine salts, process for their preparation and use thereof.

International (PCT) publication WO 2012/090043 A1 discloses solid state forms of azilsartan medoxomil and process for the preparation thereof. The solid state forms includes crystalline forms viz. J₂, J₃, J₄, J₅, J₆, J₇, J₈, J₉ and amorphous forms of azilsartan medoxomil (I).

International (PCT) publication WO 2012/119573 A1 discloses an improved method of manufacturing 2-ethoxy-1-((2′-((hydroxyamino)iminomethyl)-biphenyl-4-yl)methyl)-1H-benzo[d]imidazole-7-carboxylic acid and its esters as below

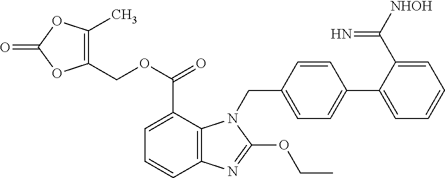

wherein R is either H or an (un)branched C₁-C₄ alkyl, ArCH₂, Ar₂CH, or Ar₃C, wherein Ar is a (un)substituted phenyl, which are suitable intermediates of synthesis of azilsartan (II), a potent antagonist of angiotensin II in ATI receptors, which is used to treat hypertension in the form of the prodrug azilsartan medoxomil (I).

International (PCT) publication WO2012/107814 A1 discloses the process for the preparation of azilsartan medoxomil (I) free from desethyl impurity. In particular, the WO '814 A1 discloses the cyclization of compound formula (3) to compound of formula (4) in presence of carbonyl source to control the formation of desethyl impurity. The WO '814 A1 also disclose the process for the preparation of 4-hydroxymethyl-5-methyl-1,3-dioxol-2-one from 4-chloromethyl-5-methyl-1,3-dioxol-2-one in presence of alkali metal iodide catalyst. To optimize the formation of 1,2,4-oxadiazol derivative compound of formula (4) and to reduce the formation of desethyl impurity, reaction was conducted under the influence of “carbonyl” source and at low temperatures.

International (PCT) publication WO 2012/157980 A2 discloses a method of manufacturing azilsartan, which treats a compound containing an amino-alcohol derivative with N,N′-carbonyldiimidazole, an inorganic base and a solvent to proceed both of cyclization and hydrolysis in a single reactor.

International (PCT) publication WO 2012/119573 A1 discloses the process for the preparation of iminomethyl compound by reacting the corresponding nitrile compound with aqueous hydroxylamine in a polar aprotic solvent, or in a mixture of polar aprotic solvents.

International (PCT) publication WO 2013/042066 A1 discloses a process for preparation of azilsartan medoxomil using 4-nitrobenzylsulfonyl chloride as a reagent and WO 2013/042067 A1 discloses process for preparation of azilsartan medoxomil potassium (I′) by using ketonic solvents.

Organic Process Research and Development (OPRD) Vol. 17 Pg. 77-86 (2013) discloses novel process for the preparation of azilsartan medoxomil (I). The new process includes transformation of the CN group into amidoxime moiety by aqueous hydroxylamine, its cyclization into the corresponding oxadiazole by treatment with dialkyl carbonates, and the following hydrolysis of the ester and transformation into the medoxomil ester. Several thus far undocumented side products were identified, and some of them were synthesized and duly characterized as potential impurities. Formation and control of possible critical impurities were also described.

International (PCT) publication WO 2012/119573 A1, WO 2012/139536 A1, WO 2012/157980 A2 and WO 2012/157977 A2 discloses the process for the preparation of azilsartan alkyl ester by cyclization in presence of base.

International (PCT) publication WO 2013/042066 A1 discloses the process for the preparation of azilsartan medoxomil (I) by formation of (2-ethoxy-1-{[2′-(5-oxo-4,5-di hydro-1,2,4-oxadiazol-3-yl)biphenyl-4-yl]methyl}-1H-benzimidazol-7-yl)-carboxyl-4-nitrophenyl sulfonate compound.

International (PCT) publication WO 2013/044816 A1 discloses crystalline forms of azilsartan, wherein the crystalline form is form A, form B, form C, form D, form E, form F, form G, form H, form I, form J or form K and process for their preparation.

International (PCT) publication WO 2013/042067 A1 (The WO '067 A1) discloses the process for the preparation of a polymorphic Form I of azilsartan medoxomil potassium (I′). The WO '067 A1 discloses preparation of Form I of azilsartan medoxomil potassium by use of C4-9ketone. The WO '067 A1 also discloses the process for the preparation of crystalline azilsartan medoxomil potassium by use of acetone in comparative example. The x-ray powder diffraction of crystalline Form I of azilsartan medoxomil potassium with that of crystalline azilsartan medoxomil potassium prepared in comparative example discloses that both the x-ray diffraction pattern as similar. Therefore, crystalline Form I may not be new form and may be obtained by use of prior art process.

EMEA study reveals that azilsartan medoxomil potassium is a white crystalline powder which is practically insoluble in water, freely soluble in methanol, dimethylsulfoxide and dimethylformamide, soluble in acetic acid, slightly soluble in acetone and acetonitrile and very slightly soluble in tetrahydrofuran and 1-octanol. It does not contain chiral center and one stable anhydrous form has been detected.

International (PCT) publication WO 2013/088384 A1 discloses crystalline and amorphous forms of azilsartan and azilsartan medoxomil potassium as well as and process for its preparation.

International (PCT) publication WO 2013/124748 A1 discloses novel crystalline forms of azilsartan medoxomil potassium and process for its preparation.

The prior art processes reported herein above discloses use of either 4-hydroxymethyl-5-methyl-1,3-dioxol-2-one or 4-chloromethyl-5-methyl-1,3-dioxol-2-one for the preparation of azilsartan medoxomil. The inventors of the present invention have found that the preparation of azilsartan medoxomil (I) results in the generation of bis-impurity (A) with respect to desired azilsartan medoxomil (I).

In view of the above, it is therefore, desirable to provide an efficient process for the preparation and purification of azilsartan medoxomil (I) contaminated with bis impurity (A) to obtain substantially pure azilsartan medoxomil (I). The present invention thereby provides useful alternative for the preparation of azilsartan medoxomil with substantial purity. Further, the present invention thereby further extends to the process for the preparation of azilsartan medoxomil potassium using purified azilsartan medoxomil (I).

### SUMMARY OF THE INVENTION

In one general aspect, there is provided an improved process for the preparation of azilsartan of Formula (II)

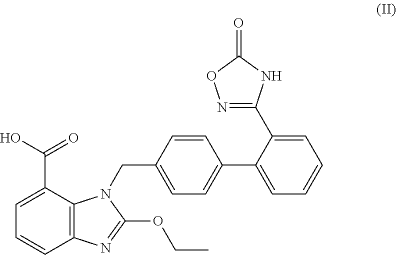 [SMILES: CCOc1nc2cccc(C(=O)O)c2n1Cc1ccc(-c2ccccc2-c2noc(=O)[nH]2)cc1]

the process comprising:
(a) reacting substituted 1-((2′-cyanobiphenyl-4-yl)methyl)-2-ethoxy-1H-benzimidazole-7-carboxylate derivative of Formula (V),

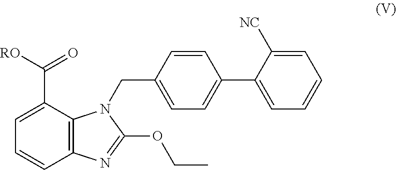

wherein R=hydrogen or C₁₋₆ alkyl
with hydroxylamine hydrochloride in one or more of suitable organic solvent in presence of base to obtain the compound of Formula (IV);

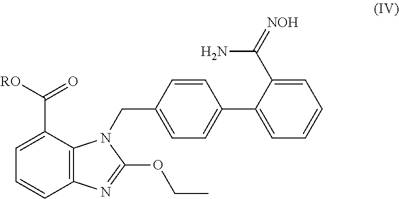

(b) reacting the compound of Formula (IV) with aryl or alkyl chloroformate in presence of base to obtain the compound of Formula (IIIA);

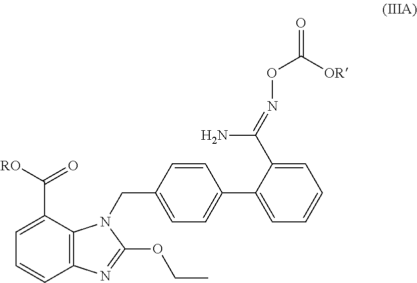

wherein R′ is a branched or unbranched, substituted or unsubstituted C₁-C₈alkyl or substituted or unsubstituted phenyl or benzyl,
(c) in-situ cyclizing the compound (IIIA) in polar solvent in absence of base to obtain compound (III); and

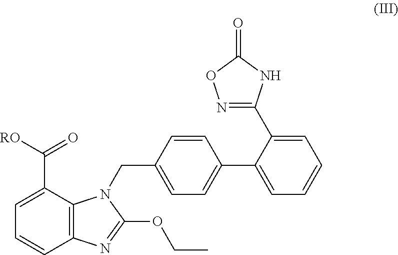

(d) hydrolyzing the compound of Formula (III) with suitable base to obtain azilsartan of Formula (II),
   wherein the improvement comprises obtaining azilsartan of Formula (II) as isopropanol solvate.
      In another general aspect, there is provided azilsartan isopropanol solvate characterized by X-ray powder diffraction, differential scanning calorimeter and theromgravimetric analysis.
      In another general aspect, there is provided an improved process for the preparation of azilsartan medoxomil of Formula (I)

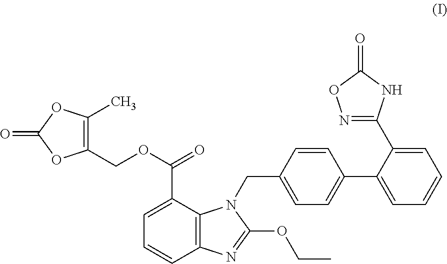 [SMILES: CCOc1nc2cccc(C(=O)OCc3oc(=O)oc3C)c2n1Cc1ccc(-c2ccccc2-c2noc(=O)[nH]2)cc1]

the process comprising:
(a) reacting substituted 1-((2′-cyanobiphenyl-4-yl)methyl)-2-ethoxy-1H-benzimidazole-7-carboxylate derivative of Formula (V),

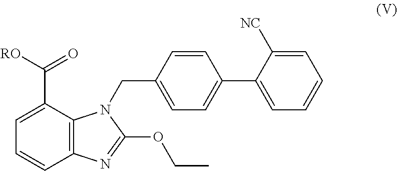

wherein R=hydrogen or C₁₋₆ alkyl
with hydroxylamine hydrochloride in one or more of suitable organic solvent in presence of base to obtain the compound of Formula (IV);

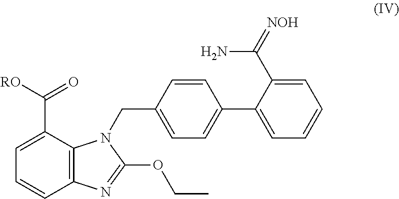

(b) reacting the compound of Formula (IV) with aryl or alkyl chloroformate in presence of base to obtain the compound of Formula (IIIA);

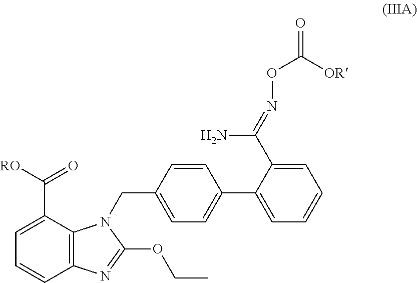

wherein R′ is a branched or unbranched, substituted or unsubstituted C₁-C₈alkyl or substituted or unsubstituted phenyl or benzyl,
(c) in-situ cyclizing the compound (IIIA) in polar aprotic solvent in absence of base to obtain compound (III); and

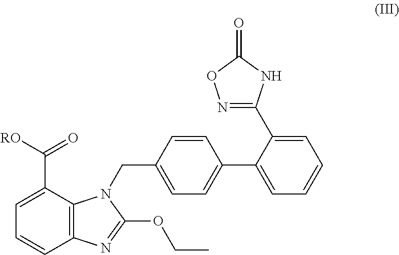

(d) hydrolyzing the compound of Formula (III) with suitable base to obtain azilsartan of Formula (II) and reacting with 4-chloromethyl-5-methyl-1,3-dioxol-2-one to obtain azilsartan medoxomil of Formula (I),
   wherein improvement comprises isolating azilsartan of Formula (II) as isopropanol solvate and azilsartan medoxomil of Formula (I) as methylene dichloride solvate.

In another general aspect, there is provided a process for purifying azilsartan medoxomil from at least one solvent selected from the group consisting of C₂₋₆ esters, a mixture of a C₂₋₆ esters and water, a mixture of a C₂₋₆ esters and N,N-dimethylformamide, a mixture of C₂₋₆ esters and N,N-dimethylacetamide, N-methylpyrrolidone, halogenated hydrocarbons, substituted or unsubstituted C₆₋₁₂aromatic hydrocarbons, dimethylsulfoxide, dimethylcarbonate, C₁₋₄ alkyl alcohols, a mixture of a C₁₋₄ alkyl alcohol and water, acetonitrile, a mixture of acetonitrile and water, C₃₋₆ ketones, a mixture of a C₃₋₆ ketones and water, ethers to obtain the purified azilsartan medoxomil, wherein the total purity of the purified azilsartan medoxomil is higher than the total purity of the starting azilsartan medoxomil.

In another general aspect, there is provided a process for the purification of azilsartan medoxomil (II)

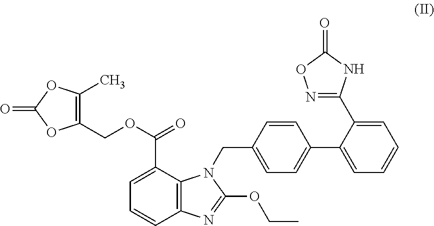 [SMILES: CCOc1nc2cccc(C(=O)OCc3oc(=O)oc3C)c2n1Cc1ccc(-c2ccccc2-c2noc(=O)[nH]2)cc1]

the process comprising:
(a) providing azilsartan medoxomil solution containing bis impurity (A) up to about 40% with respect to azilsartan medoxomil (II) in one or more of suitable organic solvent to obtain the reaction mixture;
(b) treating the reaction mixture with alkali or alkaline earth metal to obtain azilsartan medoxomil alkali metal salt (Ia);

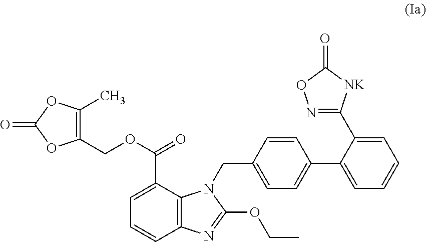

wherein M is Na, K, Li, Ca, Zn, Mg, Ba and the like,
(c) obtaining azilsartan medoxomil alkali metal salt (Ia) substantially free from bis impurity (A); and
(d) optionally converting azilsartan medoxomil alkali metal salt (Ia) to azilsartan medoxomil (II).

In another general aspect, there is provided purified azilsartan medoxomil substantially free from azilsartan (II), isopropyl ester of azilsartan (IIa), bis-impurity (A), desethyl analouge impurity (B), azilsartan methyl ester (IIIc) and azilsartan ethyl ester (IIIb).

In another general aspect, there is provided a process for purifying azilsartan medoxomil (I), the process comprising:
(a) providing azilsartan medoxomil solution containing total impurities more than 10% by area percentage of HPLC in at least one solvent selected from the group consisting of halogenated hydrocarbons, substituted or unsubstituted C₆₋₁₂ aromatic hydrocarbons, polar aprotic solvents, ethers, nitriles or mixture thereof; and
(b) crystallizing purified azilsartan medoxomil followed by removal of solvent,
   wherein the azilsartan medoxomil (I) is prepared by using 4-chloromethyl-5-methyl-1,3-dioxol-2-one.

In another general aspect, there is provided use of purified azilsartan medoxomil of Formula (I) for the preparation of azilsartan medoxomil potassium (I′).

In another general aspect, there is provided a process for the preparation of azilsartan medoxomil potassium of Formula (I′),

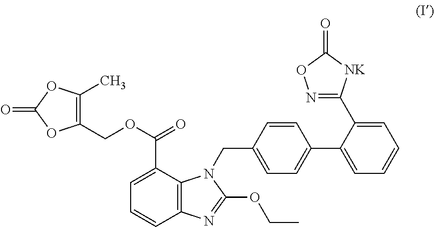

the process comprising:
(a) reacting substituted 1-((2′-cyanobiphenyl-4-yl)methyl)-2-ethoxy-1H-benzimidazole-7-carboxylate derivative of Formula (VI),

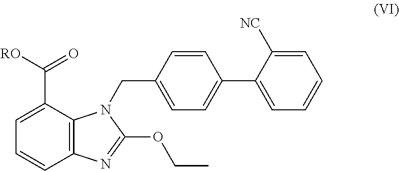

wherein R=hydrogen or C₁₋₆alkyl
with hydroxylamine hydrochloride in one or more of suitable organic solvent in presence of base to obtain the compound of Formula (IV);

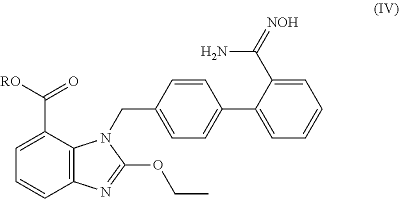

wherein R is as defined above
(b) reacting the compound of Formula (IV) with aryl or alkyl chloroformate in presence of base to obtain the compound of Formula (III);

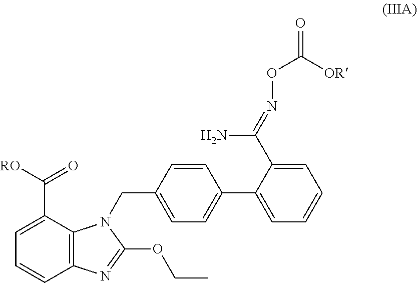

wherein R′ is a branched or unbranched, substituted or unsubstituted C₁-C₈alkyl or substituted or unsubstituted phenyl,
(c) cyclizing the compound (IIIA) in polar aprotic solvent in absence of base to obtain compound (III);

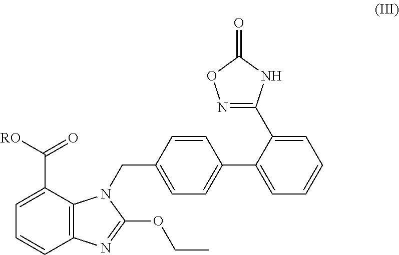

(d) hydrolyzing the compound of Formula (III) with suitable base to obtain azilsartan of Formula (II) and reacting with 4-chloromethyl-5-methyl-1,3-dioxol-2-one to obtain azilsartan medoxomil of Formula (I) with total impurities more than 10% by area percentage of HPLC;
(e) purifying the azilsartan medoxomil (I) to obtain purified azilsartan medoxomil; and
(f) converting purified azilsartan medoxomil to azilsartan medoxomil potassium (I′).

In another general aspect, there is provided use of azilsartan medoxomil solvate of Formula (I) for the preparation of azilsartan medoxomil of Formula (I′).

In another general aspect, there is provided crystalline azilsartan medoxomil potassium (I′).

In another general aspect, there is provided process for preparation of azilsartan medoxomil potassium of Formula (I′), the process comprising:
(a) dissolving purified azilsartan medoxomil methylene dichloride solvate of Formula (I) in one or more of suitable organic solvent to obtain solution;
(b) adding potassium source to the solution to obtain azilsartan medoxomil potassium in reaction mixture; and
(c) obtaining azilsartan medoxomil potassium by removal of solvent.

In another general aspect, there is provided stable azilsartan medoxomil potassium of Formula (I′).

In another general aspect, there is provided crystalline azilsartan medoxomil potassium of Formula (I′) having a HPLC purity greater than about 98%, or greater than about 99%, or greater than about 99.5%, or greater than about 99.8%, or greater than about 99.9%, as determined using high performance liquid chromatography (HPLC).

In another general aspect, there is provided pharmaceutical composition comprising therapeutically effective amount of crystalline azilsartan medoxomil potassium together with one or more pharmaceutically acceptable excipients.

### BRIEF DESCRIPTION OF THE ACCOMPANYING DRAWINGS

FIG. 1. discloses the x-ray diffractogram (XRD) of the azilsartan isopropanol solvate.

FIG. 2. discloses the differential scanning calorimetry (DSC) of the azilsartan isopropanol solvate.

FIG. 3. discloses the Thermogravimeteric Analysis (TGA) of the azilsartan isopropanol solvate.

FIG. 4. discloses the x-ray diffractogram (XRD) of the azilsartan medoxomil methylene dichloride solvate (I).

FIG. 5. discloses the differential scanning calorimetry (DSC) of the azilsartan medoxomil methylene dichloride solvate (I).

FIG. 6. discloses the Thermogravimeteric Analysis (TGA) of the azilsartan medoxomil methylene dichloride (I).

FIG. 7. discloses the x-ray diffractogram (XRD) of the azilsartan medoxomil potassium (I′).

### DETAILED DESCRIPTION OF THE INVENTION

The above and other objects of the present invention are achieved by the process of the present invention, which leads to an improved process for the preparation of crystalline azilsartan medoxomil potassium substantially free from one or more of its impurities.

The present invention can comprise (open ended) or consist essentially of the components of the present invention as well as other ingredients or elements described herein. As used herein, “comprises or comprising” means the elements recited, or their equivalent in structure or function, plus any other element or elements which are not recited.

The terms “having” and “including” are also to be construed as open ended unless the context suggest otherwise.

In general, the term “obtaining” means removal of solvent medium to obtain the product. Herein the removal of solvent may be done by a technique which includes, for example, filtration, filtration under vacuum, decantation, centrifugation, distillation and distillation under vacuum.

As used herein, the terms “starting azilsartan medoxomil” refers to azilsartan medoxomil having total purity less than 95%, particularly less than 92%, more particularly less than 90% when measured by area percentage of HPLC.

As used herein, the terms “purified azilsartan medoxomil” refers to azilsartan medoxomil having total purity greater than about 95%, particularly greater than 98%, more particularly greater than 99% when measured by area percentage of HPLC.

Optionally, the solution, prior to any solids formation, can be filtered to remove any undissolved solids, solid impurities and the like prior to removal of the solvent. Any filtration system and filtration techniques known in the art can be used.

All ranges recited herein include the endpoints, including those that recite a range “between” two values. Terms such as “about”, “generally”, “substantially,” and the like are to be construed as modifying a term or value such that it is not an absolute. This includes, at very least, the degree of expected experimental error, technique error and instrument error for a given technique used to measure a value.

As used herein, the term “stable azilsartan medoxomil potassium” refers to azilsartan medoxomil potassium that after exposure to a relative humidity of 75% at 40° C. or at 60% at 25° C., for a period of at least three months contains less than about 0.5% (wt/wt) total impurities and less than about 0.5% (wt/wt) azilsartan (II) and less than 0.15% (wt/wt) isopropyl ester of azilsartan (IIa), bis-impurity (A) and desethyl analouge impurity (B).

As used here in the term “substantially free” means
(a) azilsartan (II) impurity is present of about 0.5% or less, particularly about 0.2% or less, in azilsartan medoxomil (I) or azilsartan medoxomil potassium (I′) when measured by area percentage of HPLC;
(b) isopropyl ester of azilsartan (IIa) impurity is present of about 0.15% or less, particularly about 0.1% or less in azilsartan medoxomil (I) or azilsartan medoxomil potassium (I′) when measured by area percentage of HPLC;
(c) bis-impurity (A) is present of about 0.15% or less, particularly about 0.1% or less in azilsartan medoxomil (I) or azilsartan medoxomil potassium (I′) when measured by area percentage of HPLC;
(d) desethyl analouge impurity (B) is present of about 0.15% or less, particular about 0.1% or less in azilsartan medoxomil (I) or azilsartan medoxomil potassium (I′) when measured by area percentage of HPLC.

“Suitable solvent” means a single or a combination of two or more solvents.

As used herein, the term “crystallizing” refers to a process comprising: heating a mixture of a starting material and a solvent to a temperature of between about 40° C. and 10° C. above or below the reflux temperature of the solvent to obtain a solution, and cooling the solution to a temperature of about 0° C. to about 30° C.

In one general aspect, there is provided an improved process for the preparation of azilsartan of Formula (II)

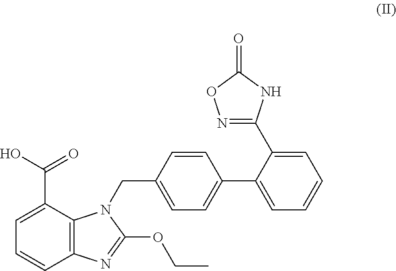 [SMILES: CCOc1nc2cccc(C(=O)O)c2n1Cc1ccc(-c2ccccc2-c2noc(=O)[nH]2)cc1]

the process comprising:
(a) reacting substituted 1-((2′-cyanobiphenyl-4-yl)methyl)-2-ethoxy-1H-benzimidazole-7-carboxylate derivative of Formula (V),

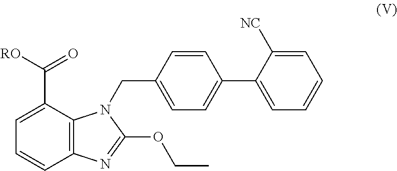

wherein R=hydrogen or C₁₋₆ alkyl
with hydroxylamine hydrochloride in one or more of suitable organic solvent in presence of base to obtain the compound of Formula (IV);

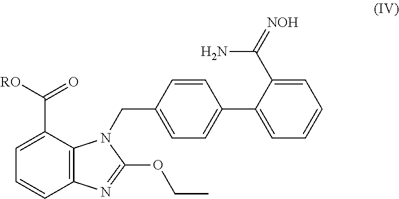

(b) reacting the compound of Formula (IV) with aryl or alkyl chloroformate in presence of base to obtain the compound of Formula (IIIA);

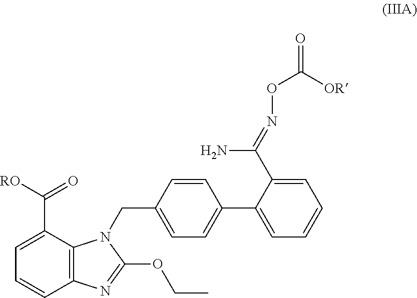

wherein R′ is a branched or unbranched, substituted or unsubstituted C₁-C₈alkyl or substituted or unsubstituted phenyl or benzyl,
(c) in-situ cyclizing the compound (IIIA) in polar solvent in absence of base to obtain compound (III); and

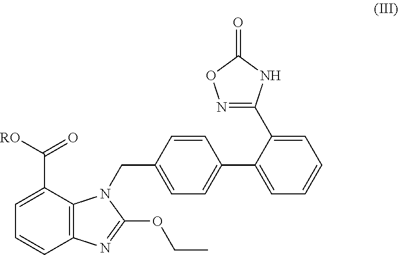

(d) hydrolyzing the compound of Formula (III) with suitable base to obtain azilsartan of Formula (II),
   wherein the improvement comprises obtaining azilsartan of Formula (II) as isopropanol solvate.
      In general, the compound substituted 1-((2′-cyanobiphenyl-4-yl)methyl)-2-ethoxy-1H-benzimidazole-7-carboxylate derivative of Formula (V) wherein R is hydrogen or C₁₋₆alkyl may be reacted with hydroxylamine hydrochloride in one or more of suitable organic solvent comprises of water, methanol, ethanol, isopropanol, n-butanol, acetone, methylethyl ketone, methylisobutyl ketone, acetonitrile, dimethylformamide, dimethylacetamide, dimethyl-sulfoxide, N-methyl pyrrolidone, tetrahydrofuran, 2-methyl tetrahydrofuran and the like. In particular, dimethylformamide, dimethylacetamide, dimethylsulfoxide, tetrahydrofuran and the like may be used to obtain the compound of Formula (IV).

In general, the suitable base for step (a) comprises of inorganic base like sodium hydroxide, potassium hydroxide, lithium hydroxide, sodium carbonate, potassium carbonate, lithium carbonate, sodium bicarbonate, potassium bicarbonate, sodium hydride, sodium methoxide, sodium ethoxide, potassium tert-butoxide and the like. In particular, sodium bicarbonate may be used.

The hydroxylamine hydrochloride is preferably anhydrous. The reaction comprises reacting compound (V) wherein R is ethyl or methyl with anhydrous hydroxylamine hydrochloride in presence of sodium bicarbonate in dimethylsulfoxide at about 70° C. to 100° C., preferably at 90° C. to 95° C. for at least 5 to about 20 hours or till completion of the reaction. The reaction mixture may be cooled to 25° C. and maintained for 1 to 5 hours.

The compound (IV) may be obtained by removal of solvent by the known techniques as disclosed herein above. The compound (IV) as a wet-cake may be slurried in water, filtered and dried to obtain compound (IV).

The embodiments of the process further includes reaction compound of Formula (IV) with aryl or alkyl chloroformate in presence of base. The suitable aryl or alkyl chloroformate comprises of phenyl chloroformate, ethyl chloroformate, propyl chloroformate, isopropyl chloroformate, isobutyl chloroformate or benzyl chloroformate and the like. In particular, ethyl chloroformate may be used to obtain the compound (IIIA).

In general, the base used for step (b) comprises of an organic base like diethylamine, triethylamine, diisopropylamine, diisopropylethylamine, pyridine, piperidine, morpholine, DBU, DABCO and the like. In particular triethylamine may be used to obtain compound of Formula (IIIA).

In general, the process comprising reacting the compound (IV) with ethylchloroformate in presence of triethylamine in one or more of suitable organic solvent comprises of water, methanol, ethanol, isopropanol, n-butanol, acetone, methylethyl ketone, methylisobutyl ketone, acetonitrile, dimethylformamide, dimethylacetamide, dimethylsulfoxide, N-methyl pyrrolidone, tetrahydrofuran, 2-methyl tetrahydrofuran, methylene dichloride, ethylene dichloride, chlorobenzene, toluene, xylene, cyclohexane and the like. In particular, methylene dichloride may be used.

The process parameter comprises maintaining the reaction mixture between 0° C. to about 20° C. for 1 hour to 5 hours or till completion of the reaction. The reaction mixture may be acidified after completion of the reaction with aqueous hydrochloric acid to adjust the pH 2-3 and thereby removal of methylene chloride from separated organic layer.

The embodiment of the process further comprises in-situ cyclizing the compound (IIIA) in polar aprotic solvent in absence of base. The polar solvent comprises one or more of water, methanol, ethanol, isopropanol, n-butanol, acetone, methyl ethyl ketone, methylisobutyl-ketone, ethyl acetate, acetonitrile, dimethylformamide, dimethylacetamide, dimethyl-sulfoxide, N-methylpyrrolidone, tetrahydrofuran, 2-methyltetrahydrofuran, formic acid, acetic acid and the like. In particular, dimethylformamide may be used.

The process improvement comprises cyclization of compound (IIIA) in absence of base. The cyclization may be performed by treating the residue obtained by removal of methylene chloride with dimethylforamide at 45-50° C. and heating the reaction mixture thus obtained at 70° C. to 120° C. for 5 to 25 hours. In particular, the reaction mixture may be heated at about 100° C. to 110° C. for 18 to 20 hours followed by cooling to 25° C. to 30° C. The reaction mixture may be further diluted with water and filtered. The obtained wet-cake may be further slurried in alcohols like methanol, ethanol, isopropanol, butanol and the like at 0° C. to 20° C.

The compound (III) may be obtained by removal of solvent by the known techniques as disclosed herein above.

The embodiments of the process further includes hydrolysis of the compound of Formula (III) in one or more of suitable solvents comprises water, alcohols, ketones, nitriles, amides. In particular, the suitable organic solvent comprises one or more of water, methanol, ethanol, isopropanol, n-butanol, acetone, methylethyl ketone, methylisobutyl ketone, acetonitrile, dimethylformamide, dimethylacetamide, dimethylsulfoxide, N-methylpyrrolidone and the like.

In particular, isopropanol may be used.

In general, the suitable base for hydrolysis comprises of inorganic base like sodium hydroxide, potassium hydroxide, lithium hydroxide, sodium carbonate, potassium carbonate lithium carbonate, sodium bicarbonate, potassium bicarbonate, sodium hydride, potassium tert-butoxide and the like. In particular, sodium hydroxide may be used.

In general, the hydrolysis of compound (III) may be performed in isopropanol with aqueous sodium hydroxide at 40° C. to about 80° C. In particular the hydrolysis may be performed at 55° C. to 60° C. for 2 hours to 8 hours, particularly for 4 hours. The reaction mixture was cooled to 25° C. and acidified with aqueous hydrochloric acid at adjust the pH 4-5. The compound (II) may be obtained by removal of solvent by the know techniques and drying.

The compound (II) thus obtained may be characterized by crystalline azilsartan isopropanol solvate. The solvate was characterized by x-ray powder diffraction, differential scanning calorimetry and thermogravimetric analysis.

The crystalline azilsartan isopropanol solvate may be characterized by x-ray powder diffraction pattern having characteristic peaks at about 7.4°, 10.9°, 18.8°, 19.7°, 21.1°, 22.0°, 22.7° and 23.1°±0.2° 2θ.

The crystalline azilsartan isopropanol solvate may further be characterized by x-ray powder diffraction pattern having characteristic peaks at about 7.4°, 8.4°, 10.9°, 13.1°, 13.5°, 14.9°, 18.3°, 18.9°, 19.7°, 21.1°, 22.0°, 22.7°, 23.1° and 24.9°±0.2° 2θ.

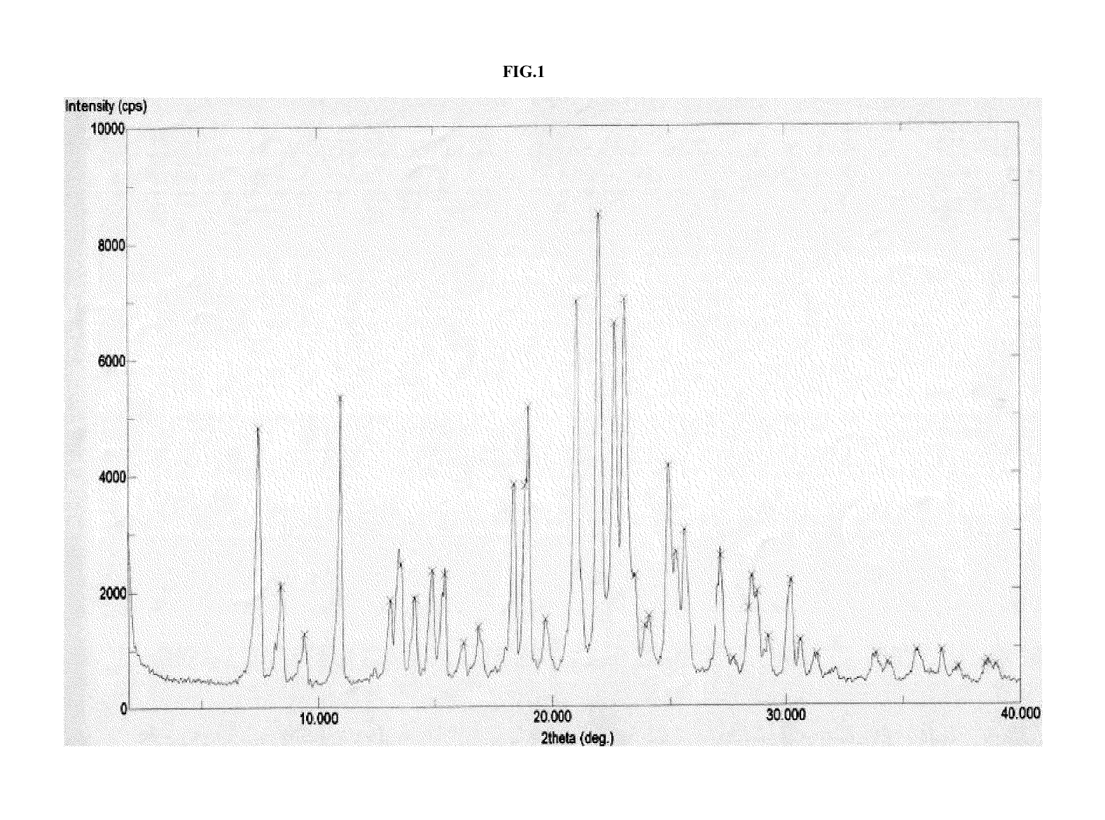

The crystalline azilsartan isopropanol solvate may further be characterized by x-ray powder diffraction pattern substantially as depicted in FIG. 1.

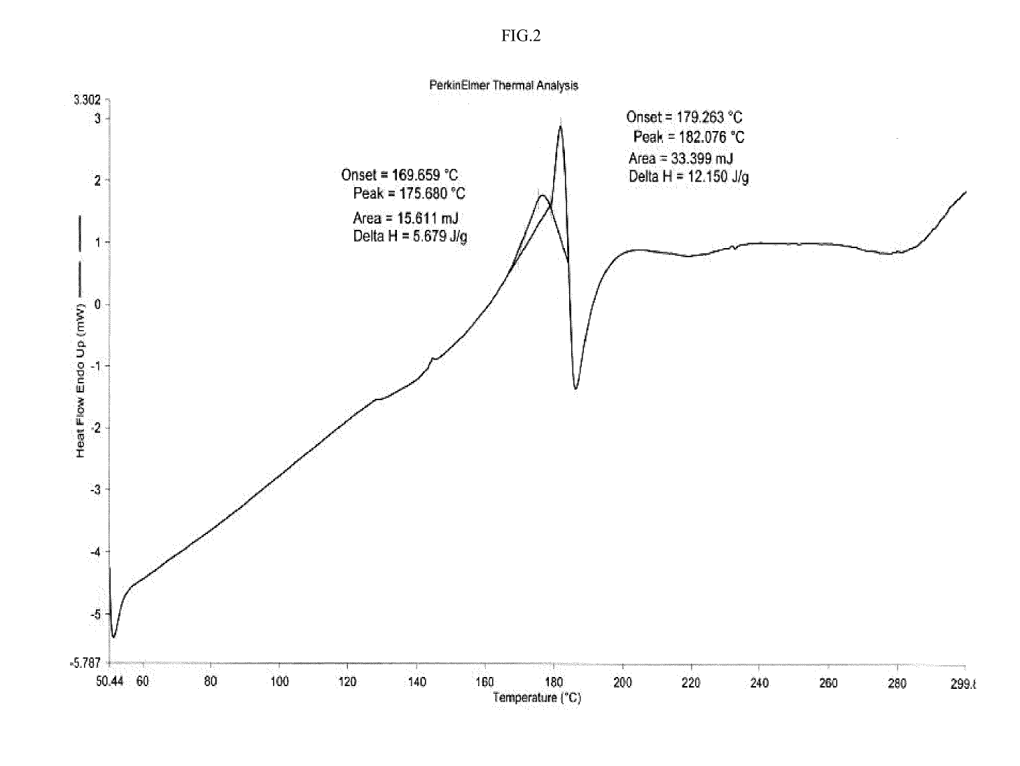

The crystalline azilsartan isopropanol solvate may be characterized by differential scanning calorimetry having endothermic peak at about 175° C.±5° C. and exothermic peak at about 182° C.±5° C. substantially as depicted in FIG. 2.

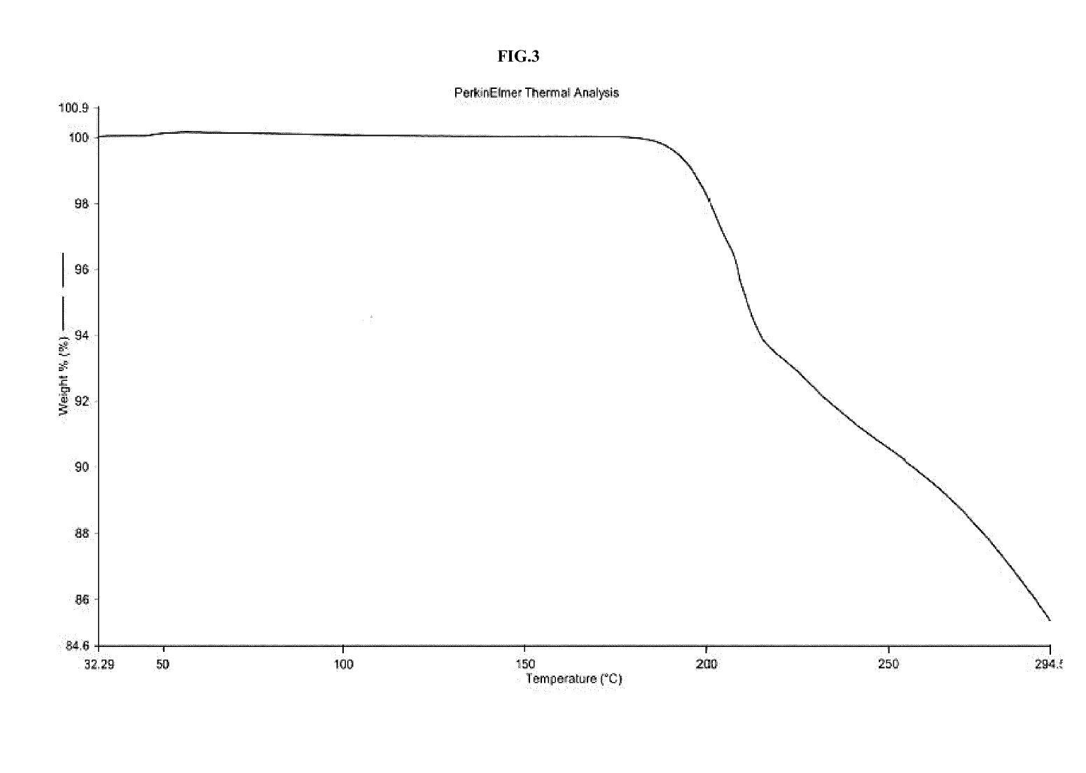

The crystalline azilsartan isopropanol solvate may be characterized by thermogravimetric analysis (TGA) as depicted in FIG. 3.

In another general aspect, there is provided an improved process for the preparation of azilsartan medoxomil of Formula (I)

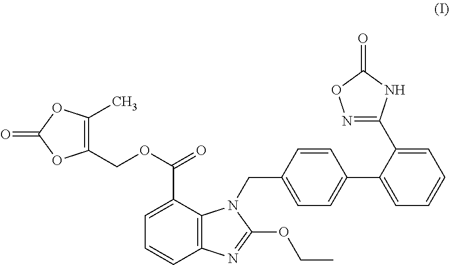 [SMILES: CCOc1nc2cccc(C(=O)OCc3oc(=O)oc3C)c2n1Cc1ccc(-c2ccccc2-c2noc(=O)[nH]2)cc1]

the process comprising:
(a) reacting substituted 1-((2′-cyanobiphenyl-4-yl)methyl)-2-ethoxy-1H-benzimidazole-7-carboxylate derivative of Formula (V),

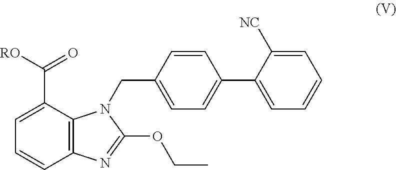

wherein R=hydrogen or C₁₋₆ alkyl
with hydroxylamine hydrochloride in one or more of suitable organic solvent in presence of base to obtain the compound of Formula (IV);

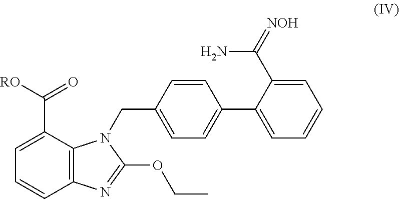

(b) reacting the compound of Formula (IV) with aryl or alkyl chloroformate in presence of base to obtain the compound of Formula (IIIA);

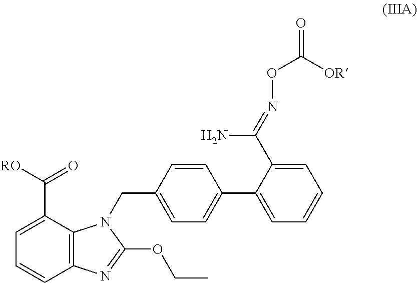

wherein R′ is a branched or unbranched, substituted or unsubstituted C₁-C₈alkyl or substituted or unsubstituted phenyl or benzyl,
(c) in-situ cyclizing the compound (IIIA) in polar aprotic solvent in absence of base to obtain compound (III);

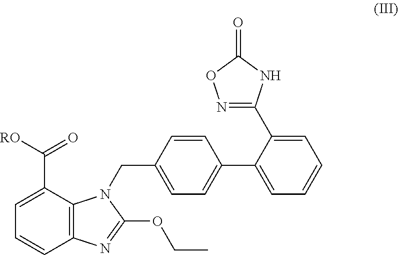

(d) hydrolyzing the compound of Formula (III) with suitable base to obtain azilsartan of Formula (II) and reacting with 4-chloromethyl-5-methyl-1,3-dioxol-2-one to obtain azilsartan medoxomil of Formula (I),
   wherein improvement comprises isolating azilsartan of Formula (II) as isopropanol solvate and azilsartan medoxomil of Formula (I) as methylene dichloride solvate.

In general, the compound (II) may be prepared by the process embodiments as disclosed herein above. The compound (II) i.e. crystalline azilsartan isopropanol solvate may be converted to azilsartan medoxomil (I) by reacting with 4-chloromethyl-5-methyl-1,3-dioxol-2-one to obtain compound of Formula (III).

The embodiment of the process comprising reacting 4-chloromethyl-5-methyl-1,3-dioxol-2-one with compound of Formula (II) in suitable polar solvent comprises one or more of water, methanol, ethanol, isopropanol, n-butanol, acetone, methyl ethyl ketone, methyl isobutylketone, ethyl acetate, acetonitrile, dimethylformamide, dimethylacetamide, dimethylsulfoxide, N-methylpyrrolidone, tetrahydrofuran, 2-methyltetrahydrofuran and the like. In particular, dimethylformamide may be used. The reaction may be performed in presence of suitable base.

In general, suitable base comprises of inorganic base like sodium hydroxide, potassium hydroxide, lithium hydroxide, sodium carbonate, potassium carbonate, lithium carbonate, cesium carbonate, sodium bicarbonate, potassium bicarbonate, sodium hydride, sodium methoxide, sodium ethoxide, potassium tert-butoxide and the like. In particular, cesium carbonate may be used.

In general, the reaction may be optionally facilitated by use of phase transfer catalyst. The suitable phase transfer catalyst comprises of tetrabutyl ammonium bromide (TBAB), tetrabutyl ammonium iodide (TBAI), benzyl triethyl ammonium chloride (TEBAC), polyethylene Glycol (PEG-200, 400, 600, 800, 1000), crown ethers like 12-crown-4, 15-crown-5, 18-crown-6, dibenzo-18-crown-6, diaza-18-crown-6 and the like.

The embodiment of the process comprises reacting isopropanol solvate of azilsartan (II) with 4-chloromethyl-5-methyl-1,3-dioxol-2-one in presence of cesium carbonate in dimethylformamide solvent at a temperature from about 25° C. to and cooling the reaction mixture. In the reaction mixture there may be addition of suitable organic solvent comprises one or more of methylene dichloride, ethylene dichloride, toluene, xylene, ethyl acetate, isopropyl acetate, butyl acetate, cyclohexane and the like. In particular, methylene dichloride may be used.

The embodiments of the process may further include removal of methylene dichloride to obtain the residue and addition of methylene dichloride again to the residue. The reaction mixture may be heated up to 60° C. and cooled to obtain azilsartan medoxomil (I) as methylene dichloride solvate.

The product azilsartan medoxomil (I) may be obtained by removal of solvent with the known techniques. The azilsartan medoxomil (I) obtained by the process is methylene dichloride solvate characterized by x-ray powder diffraction, differential scanning calorimetery and thermogravimetric analysis as disclosed herein after.

The crystalline azilsartan medoxomil (I) methylene dichloride solvate may be characterized by x-ray powder diffraction pattern having characteristic peaks at about 10.6°, 12.3°, 15.4°, 16.6°, 16.9°, 17.7°, 18.0°, 19.7°, 20.5°, 21.4°, 22.7°, 23.1°, 23.6°, 25.0° and 25.5°±0.2° 2θ.

The crystalline azilsartan medoxomil (I) methylene dichloride solvate may further be characterized by x-ray powder diffraction pattern having characteristic peaks at about 4.9°, 9.3°, 9.9°, 10.6°, 11.3°, 12.3°, 12.5°, 14.5°, 15.4°, 15.8°, 16.6°, 16.9°, 17.7°, 18.0°, 18.6°, 15.6°, 18.9°, 19.7°, 20.5°, 21.4°, 22.7°, 23.1°, 23.6°, 25.0° and 25.5°±0.2° 2θ.

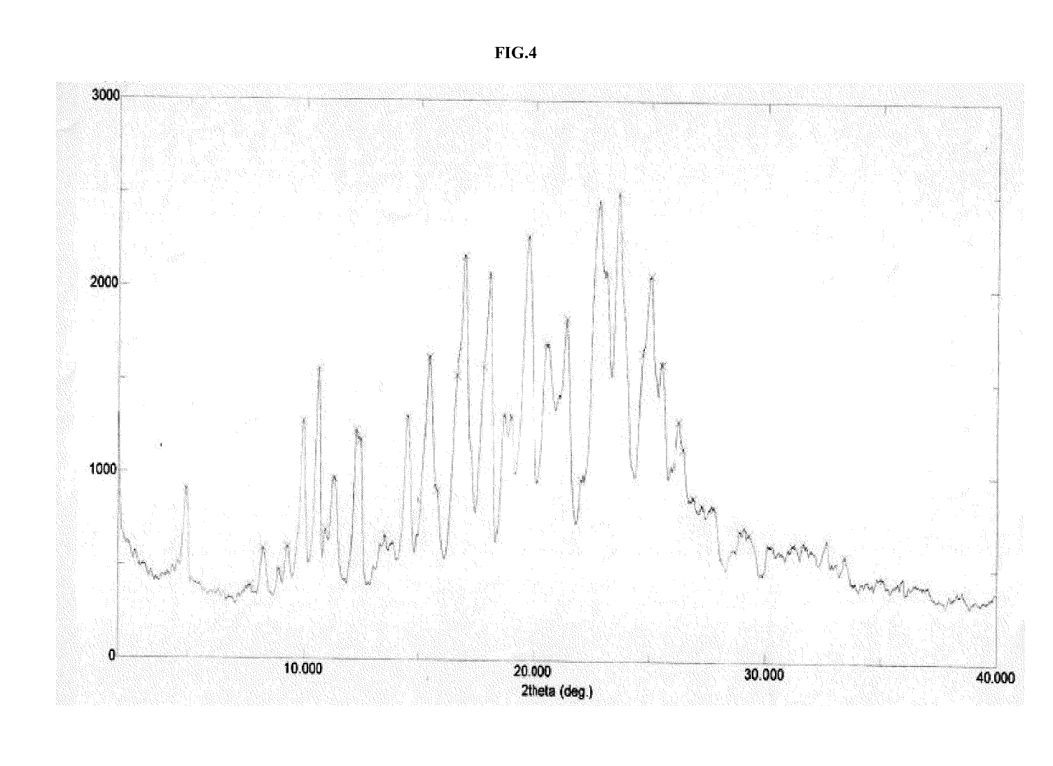

The crystalline azilsartan medoxomil (I) methylene dichloride solvate may further be characterized by x-ray powder diffraction pattern substantially as depicted in FIG. 4.

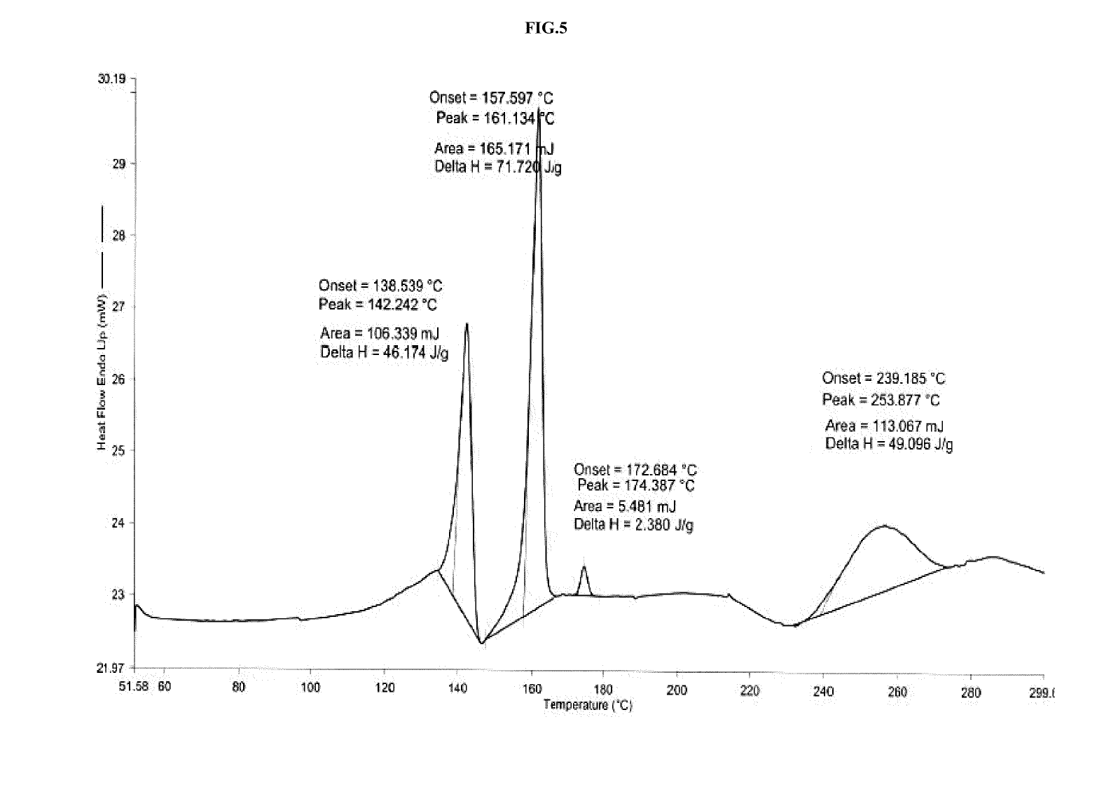

The crystalline azilsartan medoxomil (I) methylene dichloride solvate may be characterized by differential scanning calorimetry having endothermic peak at about 142° C.±5° C., 161° C.±5° C., 174° C.+5° C. and at about 253° C.±5° C. substantially as depicted in FIG. 5.

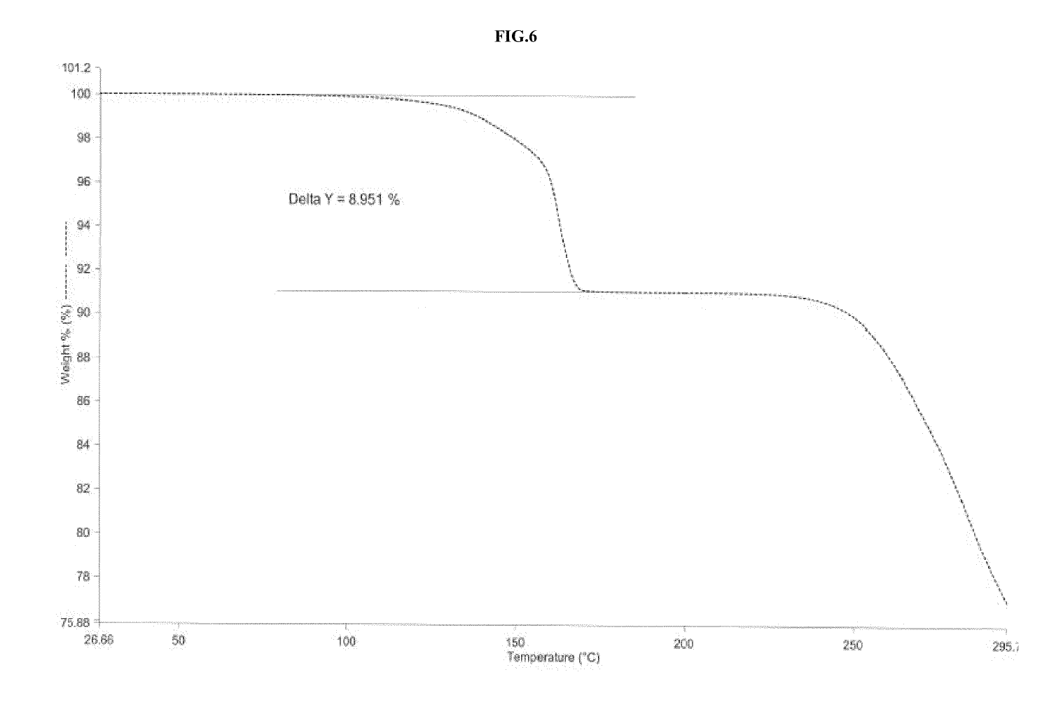

The crystalline azilsartan medoxomil (I) methylene dichloride solvate may be characterized by thermogravimetric analysis (TGA) as depicted in FIG. 6.

The azilsartan medoxomil residue obtained by the process as disclosed herein above before the treatment with methylene dichloride contains total purity less than 95%, in particular less than 92% more particular less than 90% by area percentage of HPLC. The said azilsartan medoxomil is referred as “starting azilsartan medoxomil”.

The “starting azilsartan medoxomil” contains impurities as:

azilsartan (II) from about 0.03% to about 0.15%;
isopropyl ester of azilsartan (IIa) less than about 0.1%;
bis-impurity (A) from about 20% to about 40%;
medoxomil chloride from about 5% to 10%;
desethyl analouge impurity (B) less than about 0.1%; and
Total impurities from about 35% to 45% area percentage of HPLC.

Impurity “azilsartan” is 2-ethoxy-1-((2′-(5-oxo-4,5-dihydro-1,2,4-oxadiazol-3-yl)biphenyl-4-yl)methyl)-1H-benzo[d]imidazole-7-carboxylic acid of Formula (II).

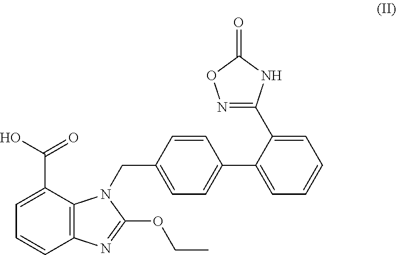 [SMILES: CCOc1nc2cccc(C(=O)O)c2n1Cc1ccc(-c2ccccc2-c2noc(=O)[nH]2)cc1]

Impurity “isopropyl ester” is isopropyl 2-ethoxy-1-((2′-(5-oxo-4,5-dihydro-1,2,4-oxadiazol-3-yl)biphenyl-4-yl)methyl)-1H-benzo[d]imidazole-7-carboxylate (IIa)

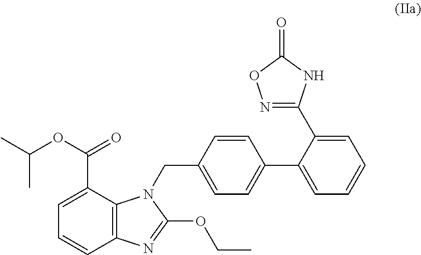 [SMILES: CCOc1nc2cccc(C(=O)OC(C)C)c2n1Cc1ccc(-c2ccccc2-c2noc(=O)[nH]2)cc1]

Impurity “bis-impurity” is (5-methyl-2-oxo-1,3-dioxol-4-yl)methyl 2-ethoxy-1-((2′-(4-((5-methyl-2-oxo-1,3-dioxol-4-yl)methyl)-5-oxo-4,5-dihydro-1,2,4-oxadiazol-3-yl)biphenyl-4-yl)methyl)-1H-benzo[d]imidazole-7-carboxylate (A)

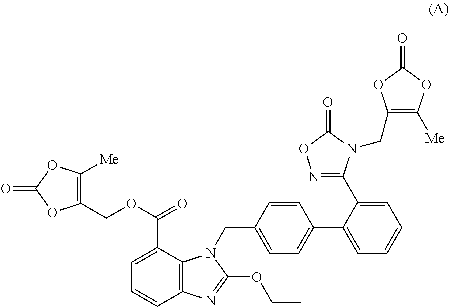 [SMILES: CCOc1nc2cccc(C(=O)OCc3oc(=O)oc3C)c2n1Cc1ccc(-c2ccccc2-c2noc(=O)n2Cc2oc(=O)oc2C)cc1]

Impurity “desethyl analagoue” is (5-methyl-2-oxo-1,3-dioxol-4-yl)methyl 2-hydroxy-1-((2′45-oxo-4,5-dihydro-1,2,4-oxadiazol-3-yl)biphenyl-4-yl)methyl)-1H-benzo[d]imidazole-7-carboxylate (B)

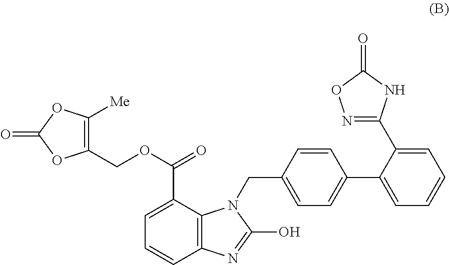 [SMILES: Cc1oc(=O)oc1COC(=O)c1cccc2nc(O)n(Cc3ccc(-c4ccccc4-c4noc(=O)[nH]4)cc3)c12]

Impurity “medoxomil chloride” is 4-chloromethyl-5-methyl-1,3-dioxol-2-one.

In another general aspect, there is provided a process for purifying azilsartan medoxomil from at least one solvent selected from the group consisting of C₂₋₆ esters, a mixture of a C₂₋₆ esters and water, a mixture of a C₂₋₆ esters and N,N-dimethylformamide, a mixture of C₁₋₆ esters and N,N-dimethylacetamide, N-methylpyrrolidone, halogenated hydrocarbons, substituted or unsubstituted C₆₋₁₂aromatic hydrocarbons, dimethylsulfoxide, dimethylcarbonate, C₁₋₄ alkyl alcohols, a mixture of a C₁₋₄ alkyl alcohol and water, acetonitrile, a mixture of acetonitrile and water, C₃₋₆ ketones, a mixture of a C₃₋₆ ketones and water, ethers to obtain the purified azilsartan medoxomil, wherein the total purity of the purified azilsartan medoxomil is higher than the total purity of the starting azilsartan medoxomil.

In general, the solvent C₂₋₆ esters comprises ethyl acetate, isopropyl acetate, butyl acetate, t-butyl acetate and like, halogenated hydrocarbons like methylene dichloride, ethylene dichloride, chloroform, chlorobenzene and the like, C₆₋₁₂ aromatic hydrocarbons like toluene, xylene, ethylbenzene and the like, C₁₋₄alkyl alcohols like methanol, ethanol, isopropanol, butanol and the like, C₃₋₆ ketones like acetone, methyl ethyl ketone, methyl isobutyl ketone and the like, ethers like diisopropyl ether, diethyl ether, methyl tert-butyl ether, 1,4-dioxane, tetrahydrofuran, 2-methyl tetrahydrofuran. In particular, methylene dichloride may be used.

The azilsartan medoxomil obtained after the reaction of isopropanol solvate of azilsartan (II) and 4-chloromethyl-5-methyl-1,3-dioxol-2-one results in the formation of bis-impurity (A) up to the level of 40% in the reaction mixture along with the formation of other impurities like desethyl analogue (B), azilsartan (II), isopropyl ester (IIa), ethyl ester of azilsartan (IIIa) and methyl ester of azilsartan (IIIb).

The azilsartan medoxomil obtained by the removal of methylene dichloride after work-up contains the impurities as disclosed herein above with total purity less than 95%, in particular less than 92% more particular less than 90% by area percentage of HPLC. The said azilsartan medoxomil is referred as “starting azilsartan medoxomil”.

Thus “starting azilsartan medoxomil” contains impurities as:

azilsartan (II) from about 0.03% to about 0.15%;
isopropyl ester of azilsartan (IIa) less than about 0.1%;
bis-impurity (A) from about 20% to about 40%;
medoxomil chloride from about 5% to 10%;
desethyl analouge impurity (B) less than about 0.1%; and
Total impurities from about 35% to 45% area percentage of HPLC.

The starting azilsartan medoxomil thus obtained may be purified from at least one solvent as disclosed herein above to obtain purified azilsartan medoxomil. In particular, the starting azilsartan medoxomil may be dissolved in methylene dichloride at about 60° C. and cooled to 25° C. The reaction mixture may be filtered and product obtained may be dried to obtain “purified azilsartan medoxomil” of Formula (I).

Thus “purified azilsartan medoxomil” contains impurities as:

azilsartan (It) from about 0.01% to about 0.03%;
isopropyl ester of azilsartan (IIa) not in detectable amount;
bis-impurity (A) from about 0.10% to about 0.12%;
medoxomil chloride not in detectable amount
desethyl analouge impurity (B) not in detectable amount; and
Total impurities from about 0.5% to 0.7% area percentage of HPLC.

In another general aspect, there is provided a process for the purification of azilsartan medoxomil (II)

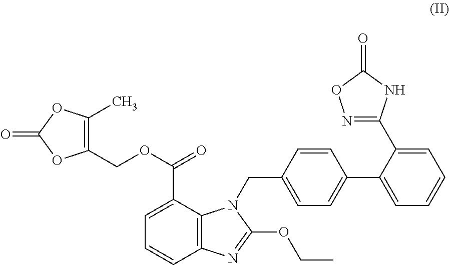 [SMILES: CCOc1nc2cccc(C(=O)OCc3oc(=O)oc3C)c2n1Cc1ccc(-c2ccccc2-c2noc(=O)[nH]2)cc1]

the process comprising:
(a) providing azilsartan medoxomil solution containing bis impurity (A) up to about 40% with respect to azilsartan medoxomil (II) in one or more of suitable organic solvent to obtain the reaction mixture;
(b) treating the reaction mixture with alkali or alkaline earth metal to obtain azilsartan medoxomil alkali metal salt (Ia);

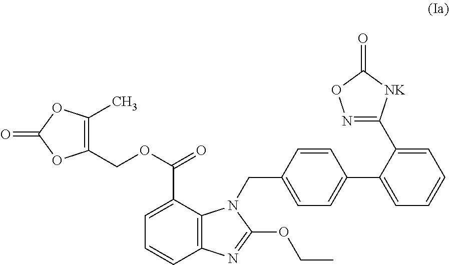

wherein M is Na, K, Li, Ca, Zn, Mg, Ba and the like,
(c) obtaining azilsartan medoxomil alkali metal salt (Ia) substantially free from bis impurity (A); and
(d) optionally converting azilsartan medoxomil alkali metal salt (Ia) to azilsartan medoxomil (II).

In general, the suitable organic solvent comprises one or more of water, methanol, ethanol, isopropanol, n-butanol, acetone, methylethyl ketone, methylisobutyl ketone, acetonitrile, dimethyl formamide, dimethyl acetamide, dimethylsulfoxide, N-methylpyrrolidone, tetrahydrofuran, 2-methyl tetrahydrofuran and the like. In particular, ketone or alcohol may be used.

The alkali metal or alkaline earth metal comprises of sodium, potassium, lithium, calcium, magnesium, zinc, barium and the like. The alkali or alkaline earth metal salt may be prepared by using suitable source of alkali or alkaline earth metal. The sources like sodium or potassium 2-ethylhexanonate may be preferred.

In another general aspect, there is provided a process for purifying azilsartan medoxomil (I), the process comprising:
(a) providing azilsartan medoxomil solution containing total impurities more than 10% by area percentage of HPLC in at least one solvent selected from the group consisting of halogenated hydrocarbons, substituted or unsubstituted C₆₋₁₂ aromatic hydrocarbons, polar aprotic solvents, ethers, nitriles or mixture thereof; and
(b) crystallizing purified azilsartan medoxomil followed by removal of solvent, wherein the azilsartan medoxomil (I) is prepared by using 4-chloromethyl-5-methyl-1,3-dioxol-2-one.

In general, the suitable solvent comprises one or more of methylene dichloride, ethylene dichloride, chlorobenzene, toluene, xylene, ethylbenzene, dimethylformamide, dimethylacetamide, dimethylcarbonate, dimethylsulfoxide, N-methylpyrrolidone, tetrahydrofuran, 2-methyl tetrahydrofuran, 1,4-dioxane, methyl tert-butyl ether, diisopropyl ether, diethylether, acetonitrile and the like. In particular, methylene dichloride may be used.

In general, the purified azilsartan medoxomil is substantially free from azilsartan (II), isopropyl ester of azilsartan (IIa), bis-impurity (A), desethyl analouge impurity (B), azilsartan methyl ester (IIIa) and azilsartan ethyl ester (IIIb).

The embodiments of the process comprises purifying the azilsartan medoxomil obtained by reaction azilsartan isopropanol solvate (II) with 4-chloromethyl-5-methyl-1,3-dioxol-2-one, wherein the total impurities is more than 10%, preferably more than 20%, more preferably more than 40% by area percentage of HPLC.

In another general aspect, there is provided azilsartan medoxomil of Formula (I) substantially free from azilsartan (II).

In another general aspect, there is provided azilsartan medoxomil of Formula (I) substantially free from isopropyl ester of azilsartan (IIa).

In another general aspect, there is provided azilsartan medoxomil of Formula (I) substantially free from bis-impurity (A).

In another general aspect, there is provided azilsartan medoxomil of Formula (I) substantially free from desethyl analouge (B).

In another general aspect, there is provided use of purified azilsartan medoxomil of Formula (I) for the preparation of azilsartan medoxomil potassium (I′).

In another general aspect, there is provided a process for the preparation of azilsartan medoxomil potassium of Formula (I′),

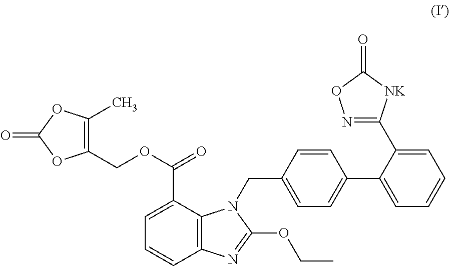

the process comprising:
(a) reacting substituted 1-((2′-cyanobiphenyl-4-yl)methyl)-2-ethoxy-1H-benzimidazole-7-carboxylate derivative of Formula (VI),

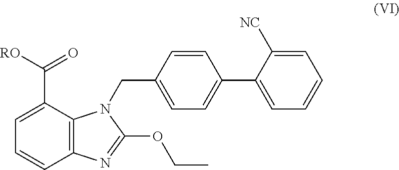

wherein R=hydrogen or C₁₋₆ alkyl
with hydroxylamine hydrochloride in one or more of suitable organic solvent in presence of base to obtain the compound of Formula (IV);

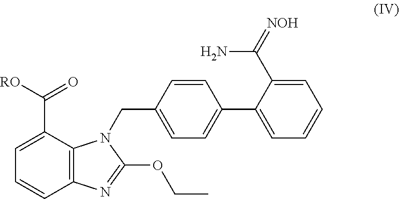

wherein R is as defined above
(b) reacting the compound of Formula (IV) with aryl or alkyl chloroformate in presence of base to obtain the compound of Formula (IIIA);

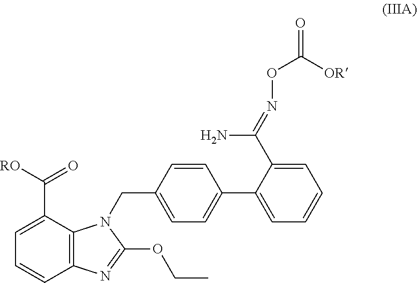

wherein R′ is a branched or unbranched, substituted or unsubstituted C₁-C₈alkyl or substituted or unsubstituted phenyl,
(c) in-situ cyclizing the compound (IIIA) in polar aprotic solvent in absence of base to obtain compound (III);

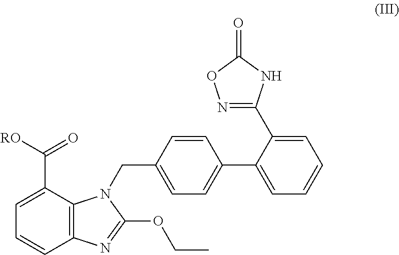

(d) hydrolyzing the compound of Formula (III) with suitable base to obtain azilsartan of Formula (II) and reacting with 4-chloromethyl-5-methyl-1,3-dioxol-2-one to obtain azilsartan medoxomil of Formula (I) with total impurities more than 10% by area percentage of HPLC;
(e) purifying the azilsartan medoxomil (I) to obtain purified azilsartan medoxomil; and
(f) converting purified azilsartan medoxomil to azilsartan medoxomil potassium (I′).

In another general aspect, there is provided a process for the preparation of azilsartan medoxomil of Formula (I),

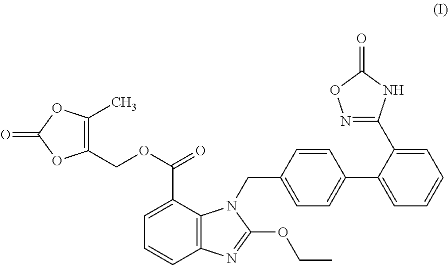 [SMILES: CCOc1nc2cccc(C(=O)OCc3oc(=O)oc3C)c2n1Cc1ccc(-c2ccccc2-c2noc(=O)[nH]2)cc1]

the process comprising:
(a) reacting substituted 1-((2′-cyanobiphenyl-4-yl)methyl)-2-ethoxy-1H-benzimidazole-7-carboxylate derivative of Formula (V),

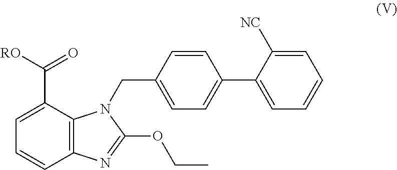

wherein R=Hydrogen or C₁₋₆alkyl
with hydroxylamine hydrochloride in one or more of suitable organic solvent in presence of base to obtain the compound of Formula (IV);

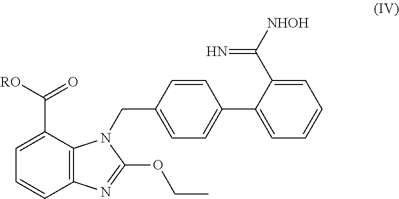

wherein R is as defined above
(b) reacting the compound of Formula (IV) with aryl or alkyl chloroformate in presence of base to obtain the compound of Formula (III);

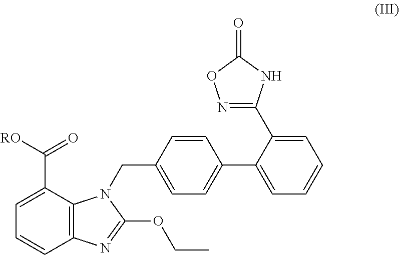

(c) hydrolyzing the compound of Formula (III) with suitable base to obtain benzimidazole-7-carboxylic acid derivative and reacting with 4-chloromethyl-5-methyl-1,3-dioxol-2-one to obtain azilsartan medoxomil of Formula (II) along with bis impurity-A;

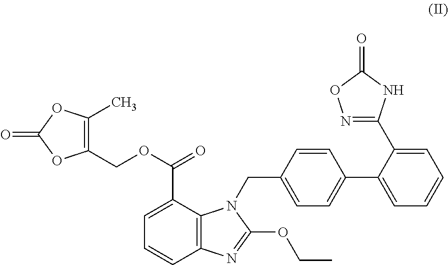 [SMILES: CCOc1nc2cccc(C(=O)OCc3oc(=O)oc3C)c2n1Cc1ccc(-c2ccccc2-c2noc(=O)[nH]2)cc1]

(d) treating azilsartan medoxomil with alkali or alkaline earth metal source to obtain alkali or alkaline earth metal salt of azilsartan medoxomil of Formula (Ia);
(e) optionally converting alkali or alkaline earth metal salt of azilsartan medoxomil to azilsartan medoxomil of Formula (II) by treating with acid; and
(f) obtaining azilsartan medoxomil of Formula (II) substantially free from bis impurity-A.

In general, the compound substituted 1-((2′-cyanobiphenyl-4-yl)methyl)-2-ethoxy-1H-benzimidazole-7-carboxylate derivative of Formula (V) may be reacted with hydroxylamine hydrochloride in one or more of suitable organic solvent selected from water, methanol, ethanol, isopropanol, n-butanol, acetone, methyl ethyl ketone, methyl isobutyl ketone, acetonitrile, dimethyl formamide, dimethyl acetamide, dimethylsulfoxide, N-methyl pyrrolidone, tetrahydrofuran, 2-methyl tetrahydrofuran and the like, In particular, dimethyl formamide, dimethyl acetamide, dimethylsulfoxide, tetrahydrofuran and the like may be used to obtain the compound of Formula (IV).

In general, the suitable base for step (a) may be selected from inorganic base like sodium hydroxide, potassium hydroxide, lithium hydroxide, sodium carbonate, potassium carbonate lithium carbonate, sodium bicarbonate, potassium bicarbonate, sodium hydride, potassium hydroxide, sodium methoxide, sodiumethoxide, potassium tert-butoxide and the like.

The embodiments of the process further includes reaction compound of Formula (IV) with aryl or alkyl chloroformate in presence of base. The suitable aryl or alkyl chloroformate may be selected from phenyl chloroformate, ethyl chloroformate, propyl chloroformate, isopropyl chloroformate, isobutyl chloroformate and the like. In particular, ethyl chloroformate may be used.

In general, the base used in step (b) may be an organic base selected from diethylamine, triethylamine, diisopropylamine, diisopropylamine, pyridine, piperidine, morpholine, DBU, DABCO and the like. In particular triethylamine may be used to obtain azilsartan medoxomil of Formula (III).

The embodiments of the process further includes hydrolysis of the compound of Formula (III) in one or more of suitable solvents selected from water, alcohols, ketones, nitriles, amides. In particular, the suitable organic solvent comprises one or more of water, methanol, ethanol, isopropanol, n-butanol, acetone, methyl ethyl ketone, methyl isobutyl ketone, acetonitrile, dimethyl formamide, dimethyl acetamide, dimethylsulfoxide, N-methyl pyrrolidone and the like. The compound (IV) obtained may be reacted in-situ with 4-chloromethyl-5-methyl-1,3-dioxol-2-one to obtain compound of Formula (III).

The said reaction results in the formation of bis-impurity-A up to the level of about 40% with respect to desired azilsartan medoxomil (I).

In general, the compound of Formula (II) having up to 40% bis-impurity A may be treated with alkali or alkaline earth metal in suitable organic solvent. The suitable organic solvent comprises from one or more of suitable organic solvent selected from water, methanol, ethanol, isopropanol, n-butanol, acetone, methyl ethyl ketone, methyl isobutyl ketone, acetonitrile, dimethyl formamide, dimethyl acetamide, dimethylsulfoxide, N-methyl pyrrolidone, tetrahydrofuran, 2-methyl tetrahydrofuran and the like. In particular, ketone or alcohol may be used.

The alkali metal or alkaline earth metal salt comprises of sodium, potassium, lithium, calcium, magnesium, zinc, barium and the like. The alkali or alkaline earth metal salt may be prepared by using suitable source of alkali or alkaline earth metal. The sources like sodium or potassium 2-ethylhexanonate may be used.

In general, alkali or alkaline earth metal salt of azilsartan medoxomil may be converted to azilsartan medoxomil (I) by treating with acid. Suitable acids may be selected from hydrochloric acid, acetic acid, nitric acid, sulfuric acid, phosphoric acid, triflouro acetic acid and the like.

The reaction may be performed in one or more of suitable solvent selected from water, alcohols, ketones, nitriles, amides. In particular, the suitable organic solvent comprises one or more of water, methanol, ethanol, isopropanol, n-butanol, acetone, methyl ethyl ketone, methyl isobutyl ketone, acetonitrile, dimethyl formamide, dimethyl acetamide, dimethylsulfoxide, N-methyl pyrrolidone and the like.

The azilsartan medoxomil of Formula (I) substantially free from bis-impurity A may be treated with potassium source to obtain azilsartan medoxomil potassium. The suitable potassium source may be potassium 2-ethylhexanoate.

In another general aspect, there is provided crystalline azilsartan medoxomil potassium (I′).

In another general aspect, there is provided a process for preparation of azilsartan medoxomil potassium of Formula (I′), the process comprising:
(a) dissolving purified azilsartan medoxomil methylene dichloride solvate of Formula (I) in one or more of suitable organic solvent to obtain solution;
(b) adding potassium source to the solution to obtain azilsartan medoxomil potassium in reaction mixture; and
(c) obtaining azilsartan medoxomil potassium (I′) by removal of solvent.

In general, the process includes dissolving azilsartan medoxomil solvate in one or more of suitable organic solvent selected from methanol, ethanol, isopropanol, n-butanol, acetone, methyl ethyl ketone, methyl isobutyl ketone, acetonitrile, dimethyl formamide, dimethyl acetamide, dimethylsulfoxide, N-methyl pyrrolidone, acetic acid, ethyl acetate, isopropyl acetate, isobutyl acetate, butyl acetate and the like. The suitable potassium source may be potassium 2-ethylhexanoate.

The azilsartan medoxomil solvate of Formula (I) is methylene dichloride solvate obtained by the process as described herein above and characterized by x-ray powder diffraction pattern having characteristic peaks at about 10.6°, 12.3°, 15.4°, 16.6°, 16.9°, 17.7°, 18.0°, 19.7°, 20.5°, 21.4°, 22.7°, 23.1°, 23.6°, 25.0° and 25.5°±0.2° 2θ.

The embodiments of the process include obtaining azilsartan medoxomil potassium by removal of solvent. The solvent may be removed by distillation under vacuum, decantation, filtration, evaporation, centrifugation and the like.

In another general aspect, there is provided a process for purification of azilsartan medoxomil potassium of Formula (I′), the process comprising:
(a) dissolving azilsartan medoxomil potassium of Formula (I′) in one or more of suitable organic solvent; and
(b) obtaining azilsartan medoxomil potassium (I′) by removal of solvent.

In general, the azilsartan medoxomil potassium of Formula (I′) may be dissolved in one or more of suitable organic solvent selected from methanol, ethanol, isopropanol, n-butanol, acetone, methyl ethyl ketone, methyl isobutyl ketone, acetonitrile, dimethyl formamide, dimethyl acetamide, dimethylsulfoxide, N-methyl pyrrolidone, acetic acid, ethyl acetate, isopropyl acetate, isobutyl acetate, butyl acetate and the like.

The azilsartan medoxomil potassium of Formula (I′) may be obtained by removal of solvent. The solvent may be removed by distillation under vacuum, decantation, filtration, evaporation, centrifugation and the like. The process may further include heating the reaction mixture containing azilsartan medoxomil potassium of Formula (I) and suitable solvent at an elevated temperature or boiling point of the solvent and cooling to an ambient temperature before removal of solvent.

The crystalline azilsartan medoxomil (I′) may be characterized by x-ray powder diffraction pattern having characteristic peaks at about 6.2°, 13.4°, 14.0°, 14.5°, 14.7°, 16.0°, 18.7°, 22.8°, 23.8° and 27.5°+0.2° 2θ.

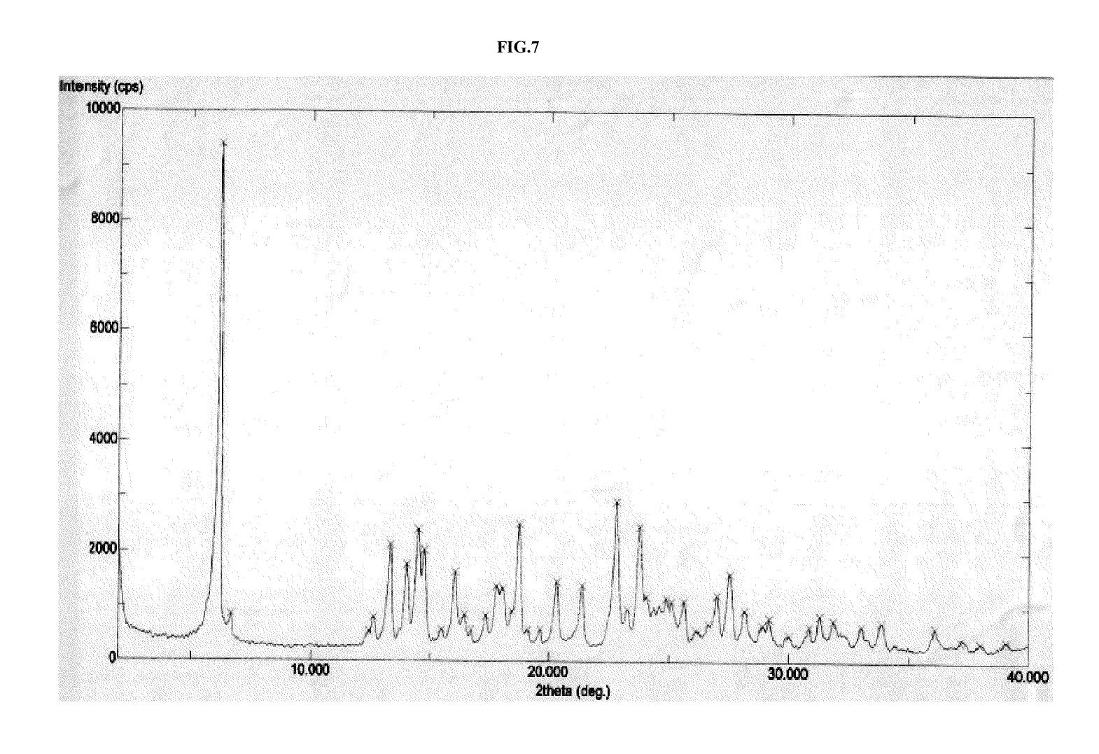

The crystalline azilsartan medoxomil (I′) may further be characterized by x-ray powder diffraction pattern substantially as depicted in FIG. 7.

In another general aspect, there is provided crystalline azilsartan medoxomil potassium of Formula (I′) having a HPLC purity of greater than about 98%, or greater than about 99%, or greater than about 99.5%, or greater than about 99.8%, or greater than about 99.9%, as determined using high performance liquid chromatography (HPLC).

In another general aspect, there is provided crystalline azilsartan medoxomil potassium having particle size distributions wherein the 10th volume percentile particle size (D10) is less than about 100 μm, the 50th volume percentile particle size (D50) is less than about 200 μm, or the 90th volume percentile particle size (D90) is less than about 400 μm, or any combination thereof. In further aspect, the crystalline azilsartan medoxomil potassium may be micronized to achieve the better particle size distribution in order to make suitable Formulation.

The active ingredient may be micronized prior to compression and shearing. Micronization may be by any suitable method. Micronization is the process of reducing the average diameter of a solid material's particles, for example by milling or grinding. In one aspect a micronized active is an active ingredient that has been subjected to a mechanical process which applies sufficient force to the active ingredient that the process is capable of breaking coarse particles down to fine particles.

In one aspect micronization of the active ingredient may be achieved using one or a combination of the following methods: ball milling, jet milling, jet blending, high-pressure homogenation, or any other milling method.

Ball milling is a milting method used in many of the prior art co-milling processes. Centrifugal and planetary ball milling may also be used.

Jet mills are capable of reducing solids to particle sizes in the low-micron to submicron range. The grinding energy is created by gas streams from horizontal grinding air nozzles. Particles in the fluidized bed created by the gas streams are accelerated towards the centre of the mill, colliding within. The gas streams and the particles carried in them create a violent turbulence and, as the particles collide with one another, they are pulverized.

Alternatively micronized active ingredient may be produced by using a high energy media mill or an agitator bead mill, for example, the Netzsch high energy media mill, or the DYNO-mill (Willy A. Bachofen AG, Switzerland).

In another general aspect, there is provided crystalline azilsartan medoxomil potassium of Formula (I′) substantially free from one or more of its impurities as determined using high performance liquid chromatography (HPLC).

In another general aspect, there is provided pharmaceutical composition comprising therapeutically effective amount of crystalline azilsartan medoxomil potassium together with one or more pharmaceutically acceptable excipients.

In another general aspect, there is provided process for the preparation of azilsartan medoxomil potassium of Formula (I′) according the reaction scheme-1 substantially as depicted herein after.

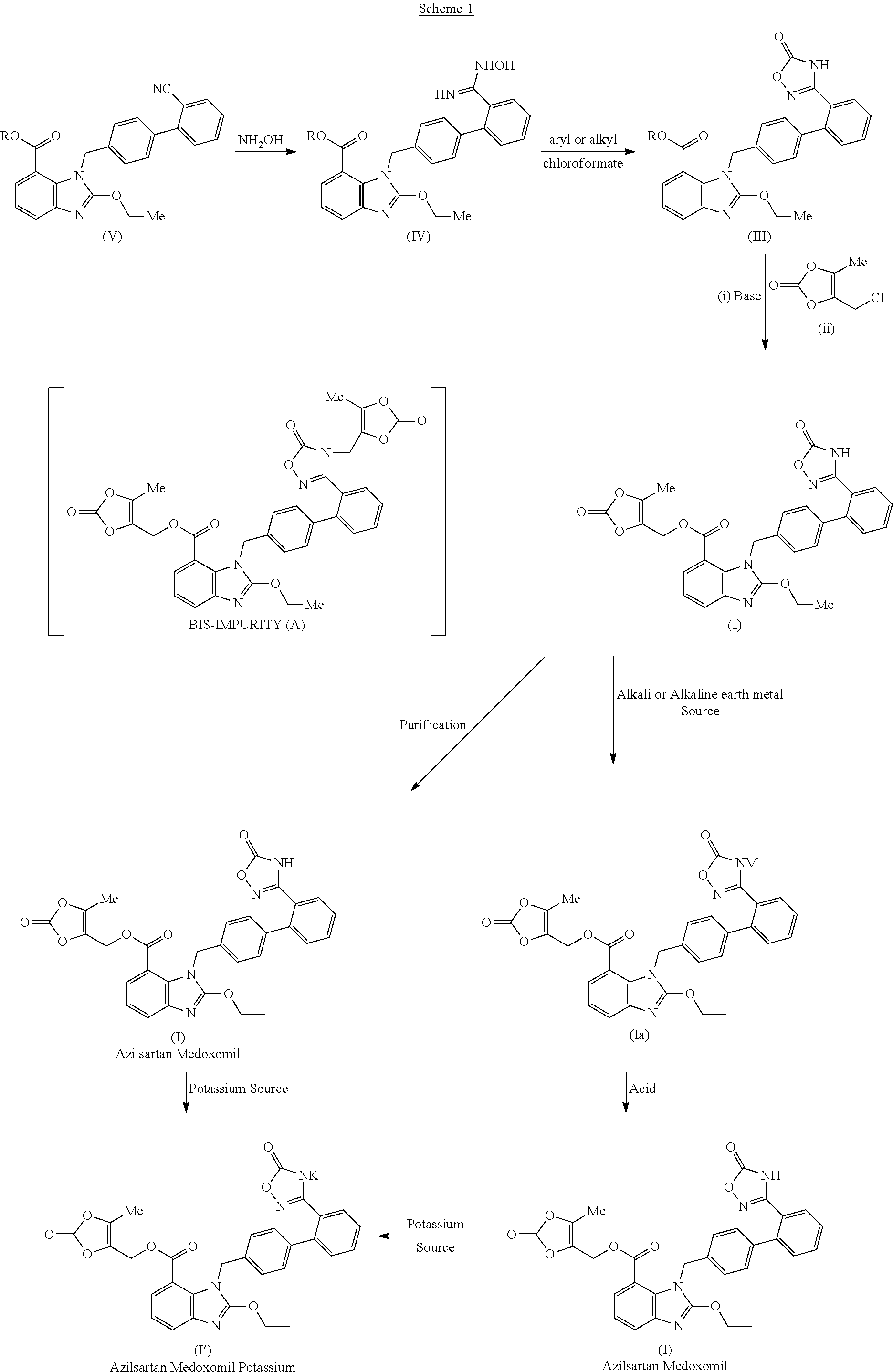

The invention also encompasses pharmaceutical compositions comprising azilsartan medoxomil potassium of the invention. As used herein, the term “pharmaceutical compositions” includes pharmaceutical Formulations like tablets, pills, powders, liquids, suspensions, emulsions, granules, capsules, suppositories, or injection preparations.

In another general aspect, there is provided pharmaceutical composition comprising therapeutically effective amount of crystalline azilsartan medoxomil potassium together with one or more pharmaceutically acceptable excipients.

In another general aspect, there is provided pharmaceutical composition comprising therapeutically effective amount of crystalline azilsartan medoxomil potassium substantially free from impurities together with one or more pharmaceutically acceptable carriers, excipients or diluents.

The examples are provided as one of the possible way to practice the invention and should not be considered as limitation of the scope of the invention.

### EXAMPLES

### Example-1

### Preparation of Imidoxime: (IV)

In a 3 liter four necked round bottom flask equipped with nitrogen atmosphere facility, mechanical stirrer, thermometer and an addition funnel, DMSO (1000 ml), sodium bicarbonate (296.1 g) and anhydrous hydroxylamine hydrochloride (163.34 g) were added and stirred for 15 min. Compound (V) (100 g) was added and the reaction mixture was heated 90° C. for 16 hours. The reaction mixture was cooled to 25-35° C. and water (1000 mL) was added and stirred for 1 hour. The product thus obtained was filtered and dried under vacuum at 80° C. for 6 hours.

### Example-2

### Preparation of Oxadiazolone (III)

In a 3 liter four necked round bottom flask equipped with nitrogen atmosphere facility, mechanical stirrer, thermometer and an addition funnel, imidoxime (IV) as obtained in example 1 and methylene dichloride (485 ml), triethylamine (27.84 g) were added at 25° C. and reaction mixture was cooled to 0-5° C. Ethyl chloroformate solution (27.55 g) in 48.5 ml methylene dichloride was added at 0-5° C. and stirred for 30 min. The reaction mixture was stirred for 1 hour at 15-20° C. and water (291 ml) was added. The conc. hydrochloric acid was added to adjust the pH 2-3 and thereby organic layer was separated. The methylene dichloride layer was washed with water and dried over anhydrous sodium sulphate. The organic layer was filtered and distilled under reduced pressure. Dimethylformamide (485 ml) was added at 45-50° C. and further heated to 100-110° C. for 18 hours. The reaction mixture was cooled to 25-30° C., diluted with water (970 ml) and stirred for 2 hours. The reaction mixture was and washed with water. The wet-cake was slurried in mixture of water (194 ml) and methanol (485 ml) and heated to reflux for 1 hour and cooled to 0-5° C. The product was filtered and washed with methanol and dried under vacuum at 80° C. for 6 hours.

### Example-3

### Preparation of Azilsartan (II)

In a 3 liter four necked round bottom flask equipped with nitrogen atmosphere facility, mechanical stirrer, thermometer and an addition funnel, isopropanol (340 ml) and oxazolidinone (III) (68 g) as obtained in example-2 were added and the reaction mixture was stirred for 15 min at 25-30° C. Sodium hydroxide solution (14.04 g) in 68 ml water was added and heated to 55-60° C. for 4 hours. The reaction mixture was cooled to 20-25° C. and 408 ml of water was added. The pH of the reaction mixture was adjusted to 4-5 using hydrochloric acid and stirred for 1 hour. The product was filtered and wet-cake was washed with isopropanol. The product was dried under vacuum at 80° C. for 6 hours to obtain crystalline isopropanol solvate of azilsartan (II). The product was characterized by x-ray powder diffraction (FIG. 1), differential scanning calorimetry (FIG. 2) and thermogravimetric analysis (FIG. 3).

### Example-4

### Preparation of Azilsartan Medoxomil (I)

In a 1 liter four necked round bottomed flask equipped with nitrogen atmosphere facility, mechanical stirrer, thermometer and an addition funnel, azilsartan isopropanol solvate (II) (200 g) was dissolved in dimethylformamide (1.6 L). Cesium carbonate (157 g) and 4-chloromethyl-5-methyl-1,3-dioxol-2-one (78.1 g) in dimethylformamide (300 mL) solution was added and the mixture was stirred at room temperature for 6 hours. After the completion of the reaction as monitored by HPLC, the reaction mixture was cooled to 10° C. to 20° C. Water (3 L) and methylene dichloride (3 L) were added. The reaction mixture was acidified with 15% hydrochloric acid to adjust the pH 4-5. The organic layer was separated and treated with 5% sodium bicarbonate. The organic layer was washed with water and dried over anhydrous sodium sulfate and distilled to remove methylene dichloride and obtain azilsartan medoxomil. The product was analyzed by area percentage of HPLC.

### Impurity Profile:

azilsartan (II) from about 0.03% to about 0.15%;
isopropyl ester of azilsartan (IIa) less than about 0.1%;
bis-impurity (A) from about 20% to about 40%;
medoxomil chloride from about 5% to 10%;
desethyl analouge impurity (B) less than about 0.1%; and
Total impurities from about 35% to 45% area percentage of HPLC.

### Purification of Azilsartan Medoxomil (I):

In a 1 liter four necked round bottomed flask equipped with nitrogen atmosphere facility, mechanical stirrer, thermometer and an addition funnel, azilsartan medoxomil obtained above was treated with methylene dichloride (600 mL) and heated to 40° C. to 45° C. for 1 hour. The reaction mixture was cooled to 0° C. to 5° C. and stirred for 1 hour. The product was filtered and washed with methylene dichloride. The product was dried under vacuum at 70° C. for 6 hours to obtain purified azilsartan medoxomil as crystalline methylene dichloride solvate. The product was characterized by x-ray powder diffraction (FIG. 4), differential scanning calorimetry (FIG. 5) and thermogravimetric analysis (FIG. 6).

The product was analyzed by area percentage of HPLC.

### Impurity Profile:

azilsartan (II) from about 0.01% to about 0.03%;
isopropyl ester of azilsartan (IIa) not in detectable amount;
bis-impurity (A) from about 0.10% to about 0.12%;
medoxomil chloride not in detectable amount
desethyl analouge impurity (B) not in detectable amount; and
Total impurities from about 0.5% to 0.7% area percentage of HPLC.

### Example-5

### Purification of Azilsartan Medoxomil of Formula (I)

In a 1 liter four necked round bottomed flask equipped with nitrogen atmosphere facility, mechanical stirrer, thermometer and an addition funnel, the azilsartan medoxomil obtained above having bis-impurity (A) up to the level of 30% was heated to 55° C. in acetone. Potassium 2-ethylhexanonate (1 g) in acetone (12 mL) was added and stirred for 30 minutes. The reaction mixture cooled to 5° C. and stirred for 3 hours. The reaction mixture was distilled under vacuum at 25° C. and the residue was treated with THF (30 mL). The reaction mixture was filtered and washed with THF to obtain azilsartan medoxomil potassium (I′) with bis-impurity (A) less than 1.0% and less than 2.0% of azilsartan (II). The purity of azilsartan medoxomil potassium (I′) was at least 97% by HPLC.

### Example-6

### Preparation of Azilsartan Medoxomil Potassium (I′)

In a 1 liter four necked round bottomed flask equipped with nitrogen atmosphere facility, mechanical stirrer, thermometer and an addition funnel, acetone (1.22 L) and purified azilsartan medoxomil methylene dichloride solvate (94 g) as obtained in example-4 were added. The reaction mixture was heated to 50-55° C. and stirred for 15 min. Potassium 2-ethylhexanoate solution (30.14 g) in 94 mL acetone was added and stirred for 15 min. The reaction mass was cooled to 25-30° C. and stirred for 1 hour. The product was filtered and wet cake was washed with acetone. The product was dried under vacuum at 60° C. for 6 hours to obtain crystalline azilsartan medoxomil potassium (I′). X-ray powder diffraction (FIG. 7).

The product was analyzed by area percentage of HPLC.

### Impurity Profile:

azilsartan (II) from about 0.07% to about 0.13%;
isopropyl ester of azilsartan (IIa) not in detectable amount;
bis-impurity (A) from about 0.01% to about 0.03%;
medoxomil chloride from about 0.07% to about 0.12%
desethyl analouge impurity (B) from about 0.01% to about 0.03%; and
Total impurities less than 0.3% by area percentage of HPLC.

The purity of azilsartan medoxomil (I) or azilsartan medoxomil potassium (I′) was performed by using following HPLC conditions.
Column: waters symmetry C-18 (150×4.6) mm,×5 μm
Mobile Phase-A: Buffer
Mobile Phase-B: ACN
Wavelength: 225 nm
Column oven temp.: 30° C.
Injection volume: 10 μl
Flow rate: 1.0 ml/min
   Run time: 40 min

### Sample Temperature: 5° C.

Diluent: ACN (For azilsartan medoxomil Potassium (I′))
ACN:Water (90:10) (For azilsartan medoxomil (I))

### Gradient Programme:

| Time (min) | % Buffer | % ACN |
|---|---|---|
| 0.01 | 60 | 40 |
| 5 | 60 | 40 |
| 15 | 30 | 70 |
| 30 | 30 | 70 |
| 35 | 60 | 40 |
| 40 | 60 | 40 |

### Example-7

### Preparation of Azilsartan Medoxomil Potassium of Formula (I′)

In a 250 mL four necked round bottomed flask equipped with nitrogen atmosphere facility, mechanical stirrer, thermometer and an addition funnel, purified azilsartan medoxomil methylene dichloride solvate (5 g) obtained in example 4 was dissolved in methanol (50 mL) at 50° C. The solution was ice-cooled and a solution of potassium 2-ethylhexanoate (1.5 g) in methanol (20 mL) was added dropwise. The reaction mixture was heated to reflux and cooled. Water (50 mL) was added to the reaction mixture to precipitate azilsartan medoxomil potassium (I′). The precipitated crystals were collected by filtration and dried under reduced pressure at 60° C. for 6 hours to obtain azilsartan medoxomil potassium. (2.5 g, 50%).

### Example-8

### Purification of Azilsartan Medoxomil Potassium of Formula (I)

In a 250 mL four necked round bottomed flask equipped with nitrogen atmosphere facility, mechanical stirrer, thermometer and an addition funnel, azilsartan medoxomil potassium (5 g) obtained in example 6 was dissolved in acetone (50 mL) at 70° C. The solution was stirred for 4 hours and cooled to ambient temperature. The precipitated crystals were filtered and washed with acetone to give azilsartan medoxomil potassium (2.5 g, 50%) having purity greater than 99.5% by HPLC.

While the present invention has been described in terms of its specific embodiments, certain modifications and equivalents will be apparent to those skilled in the art and are intended to be included within the scope of the present invention.

1-31. (canceled)

32. An improved process for the preparation of azilsartan medoxomil of Formula (I) or its potassium salt of Formula (I′) 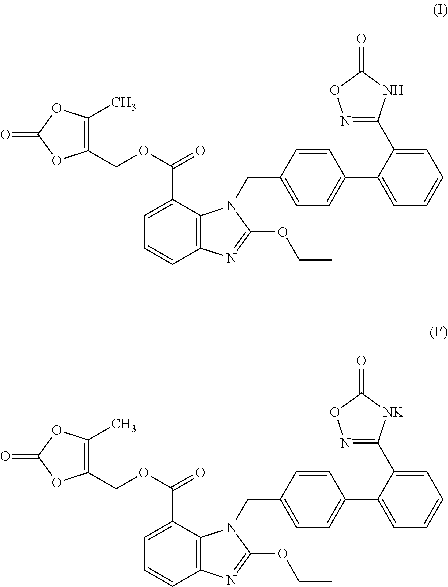
the process comprising:
(a) reacting substituted 1-((2′-cyanobiphenyl-4-yl)methyl)-2-ethoxy-1H-benzimidazole-7-carboxylate derivative of Formula (V), 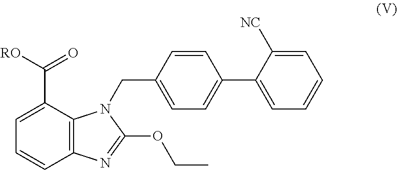
wherein R=hydrogen or C₁₋₆ alkyl
with hydroxylamine hydrochloride in one or more of suitable organic solvents in presence of base to obtain the compound of Formula (IV); 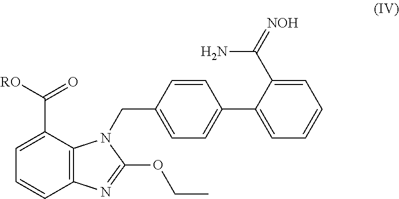
(b) reacting the compound of Formula (IV) with aryl or alkyl chloroformate in presence of base to obtain the compound of Formula (IIIA); 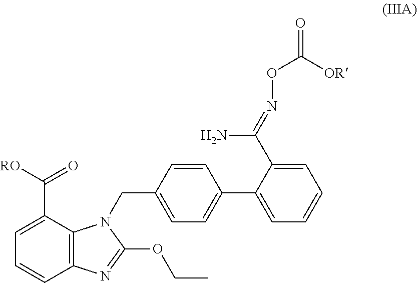
wherein R′ is a branched or unbranched, substituted or unsubstituted C₁-C₈alkyl or substituted or unsubstituted phenyl or benzyl,
(c) in-situ cyclizing the compound (IIIA) in polar aprotic solvent in absence of base to obtain compound (III); 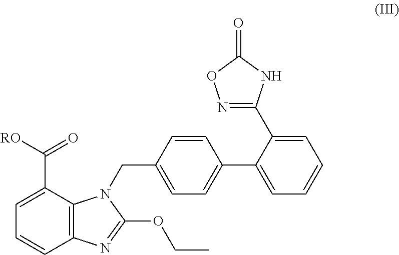
(d) hydrolyzing the compound of Formula (III) with suitable base to obtain azilsartan of Formula (II) 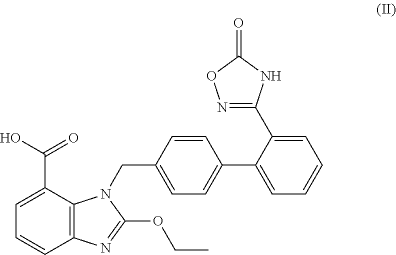 [SMILES: CCOc1nc2cccc(C(=O)O)c2n1Cc1ccc(-c2ccccc2-c2noc(=O)[nH]2)cc1]
and reacting with 4-chloromethyl-5-methyl-1,3-dioxol-2-one to obtain azilsartan medoxomil of Formula (I); and
(e) optionally converting azilsartan medoxomil of Formula (I) to its potassium salt of Formula (I′),
wherein improvement comprises isolating azilsartan of Formula (II) as isopropanol solvate and azilsartan medoxomil of Formula (I) as methylene dichloride solvate.

33. The process according to claim 32, wherein suitable organic solvent of step (a) is selected from water, methanol, ethanol, isopropanol, n-butanol, acetone, methylethyl ketone, methylisobutyl ketone, acetonitrile, dimethylformamide, dimethylacetamide, dimethyl-sulfoxide, N-methyl pyrrolidone, tetrahydrofuran, and 2-methyl tetrahydrofuran or a mixture thereof.

34. The process according to claim 32, wherein the base of step (b) comprises an inorganic base.

35. The process according to claim 34, wherein the inorganic base is selected from sodium hydroxide, potassium hydroxide, lithium hydroxide, sodium carbonate, potassium carbonate, lithium carbonate, sodium bicarbonate, potassium bicarbonate, sodium hydride, sodium methoxide, sodium ethoxide, potassium tert-butoxide or a mixture thereof.

36. The process according to claim 32, wherein suitable aryl or alkyl chloroformate is selected from phenyl chloroformate, ethyl chloroformate, propyl chloroformate, isopropyl chloroformate, isobutyl chloroformate and benzyl chloroformate or a mixture thereof.

37. The process according to claim 32, wherein in step (b) the base comprises an organic base.

38. The process according to claim 32, wherein the organic based is selected from diethylamine, triethylamine, diisopropylamine, diisopropylethylamine, pyridine, piperidine, morpholine, DBU, and DABCO or a mixture thereof.

39. The process according to claim 32, wherein in step (c) the suitable polar aprotic solvent comprises one or more of water, methanol, ethanol, isopropanol, n-butanol, acetone, methyl ethyl ketone, methylisobutylketone, ethyl acetate, acetonitrile, dimethylformamide, dimethylacetamide, dimethylsulfoxide, N-methylpyrrolidone, tetrahydrofuran, 2-methyl tetrahydrofuran, formic acid, and acetic acid.

40. The process according to claim 32, wherein in step (d) the base comprises an inorganic base selected from sodium hydroxide, potassium hydroxide, lithium hydroxide, sodium carbonate, potassium carbonate lithium carbonate, sodium bicarbonate, potassium bicarbonate, sodium hydride, and potassium tert-butoxide.

41. A crystalline azilsartan isopropanol solvate characterized by x-ray powder diffraction pattern having characteristic peaks at about 7.4°, 10.9°, 18.8°, 19.7°, 21.1°, 22.0°, 22.7° and 23.1°±0.2° 2θ.

42. A crystalline azilsartan medoxomil (I) methylene dichloride solvate characterized by x-ray powder diffraction pattern having characteristic peaks at about 10.6°, 12.3°, 15.4°, 16.6°, 16.9°, 17.7°, 18.0°, 19.7°, 20.5°, 21.4°, 22.7°, 23.1°, 23.6°, 25.0° and 25.5°±0.2° 2θ.

43. A process for the purification of azilsartan medoxomil (I) 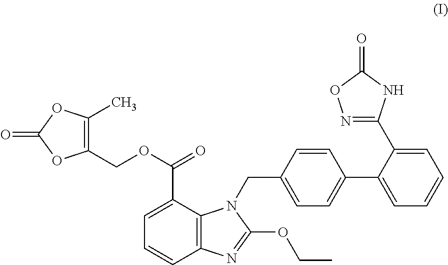 [SMILES: CCOc1nc2cccc(C(=O)OCc3oc(=O)oc3C)c2n1Cc1ccc(-c2ccccc2-c2noc(=O)[nH]2)cc1]
the process comprising:
(a) providing azilsartan medoxomil solution containing bis impurity (A) up to about 40% with respect to azilsartan medoxomil (I) in one or more of suitable organic solvent to obtain the reaction mixture;
(b) treating the reaction mixture with alkali or alkaline earth metal to obtain azilsartan medoxomil alkali metal salt (Ia); 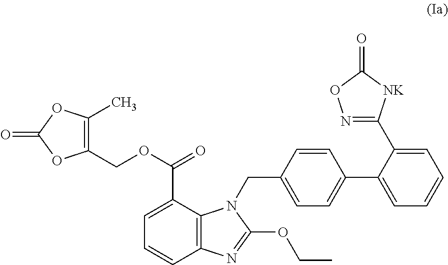
wherein M is Na, K, Li, Ca, Zn, Mg, Ba and the like,
(c) obtaining azilsartan medoxomil alkali metal salt (Ia) substantially free from bis impurity (A); and
(d) optionally converting azilsartan medoxomil alkali metal salt (Ia) to azilsartan medoxomil (I).

44. The process according to claim 43, wherein suitable organic solvent comprises one or more of water, methanol, ethanol, isopropanol, n-butanol, acetone, methylethyl ketone, methylisobutyl ketone, acetonitrile, dimethyl formamide, dimethyl acetamide, dimethylsulfoxide, N-methylpyrrolidone, tetrahydrofuran, and 2-methyl tetrahydrofuran.

45. The process according to claim 43, wherein alkali metal or alkaline earth metal is prepared by a suitable source of alkali or alkaline earth metal comprising sodium or potassium 2-ethylhexanoate.

46. A process for preparing azilsartan medoxomil (I) or its pharmaceutically acceptable potassium salt of Formula (I′), the process comprising either of:
(a) providing azilsartan medoxomil solution containing total impurities more than 10% by area percentage of HPLC in at least one solvent selected from the group consisting of halogenated hydrocarbons, substituted or unsubstituted C₆₋₁₂ aromatic hydrocarbons, polar aprotic solvents, ethers, nitriles or mixture thereof; and
(b) obtaining azilsartan medoxomil (I) by removal of solvent,
wherein the azilsartan medoxomil (I) is preparing by using 4-chloromethyl-5-methyl-1,3-dioxol-2-one; OR
(a) dissolving azilsartan medoxomil methylene dichloride solvate in one or more of suitable organic solvents to obtain solution;
(b) adding potassium source to the solution to obtain azilsartan medoxomil potassium in reaction mixture; and
(c) obtaining azilsartan medoxomil potassium (I′) by removal of solvent.

47. The process according to claim 46, wherein the suitable solvent comprises one or more of methylene dichloride, ethylene dichloride, chlorobenzene, toluene, xylene, ethylbenzene, dimethylformamide, dimethylacetamide, dimethylcarbonate, dimethylsulfoxide, N-methyl pyrrolidone, tetrahydrofuran, 2-methyl tetrahydrofuran, 1,4-dioxane, methyl tert-butyl ether, diisopropyl ether, diethylether, and acetonitrile.

48. The process according to claim 46, wherein purified azilsartan medoxomil is substantially free from azilsartan (II), isopropyl ester of azilsartan (IIa), bis-impurity (A), desethyl analogue impurity (B), azilsartan methyl ester (IIIa) and azilsartan ethyl ester (IIIb).

49. The process according to claim 46, wherein suitable solvent organic solvent comprises one or more of methanol, ethanol, isopropanol, n-butanol, acetone, methyl ethyl ketone, methyl isobutyl, ketone, acetonitrile, dimethyl formamide, dimethyl acetamide, dimethylsulfoxide, N-methyl pyrrolidone, acetic acid, ethyl acetate, isopropyl acetate, isobutyl acetate, and butyl acetate.

50. The process according to claim 46, wherein obtained azilsartan medoxomil potassium of Formula (I′) is stable after exposure to a relative humidity of 75% at 40° C. or 60% at 25° C., for a period of at least three months that contains less than about 0.5% (wt/wt) total impurities and less than about 0.5% (wt/wt) azilsartan (II) and less than 0.15% (wt/wt) isopropyl ester of azilsartan (IIa), bis-impurity (A) and desethyl analogue impurity (B).

51. A pharmaceutical composition comprising therapeutically effective amount of crystalline azilsartan medoxomil potassium obtained by the process according to claim 46, substantially free from impurities together with one or more pharmaceutically acceptable carriers, excipients or diluents.
